SEQUENCE LISTING (52 sequences)

SEQ ID NO: 1 - protein, 107 aa, Artificial Sequence [sequence omitted]

SEQ ID NO: 2 - protein, 107 aa, Artificial Sequence
  VL2
DIQMTQSPSSLSASVGDRVTITCRASQDVNIAVAWYQQKPGKAPKLLIYSASFLYSGVPSRFSGSRSGTDFTLTISSLQPEDFATYYCQQHYTTPPTFGQGTKVEIK

SEQ ID NO: 3 - protein, 107 aa, Artificial Sequence
  VL3
DIQMTQSPSSLSASVGDRVTITCRASQDVNTAVAWYQQKPGKAPKLLIYSASFLYSGVPSRFSGSRSGTDFTLTISSLQPEDFATYYCQQHYTYPPTFGQGTKVEIK

SEQ ID NO: 4 - protein, 107 aa, Artificial Sequence
  VL4
DIQMTQSPSSLSASVGDRVTITCRASQDVNIAVAWYQQKPGKAPKLLIYSASFLYSGVPSRFSGSRSGTDFTLTISSLQPEDFATYYCQQHYTYPPTFGQGTKVEIK

SEQ ID NO: 5 - protein, 107 aa, Artificial Sequence [sequence omitted]

SEQ ID NO: 6 - protein, 107 aa, Artificial Sequence
  VL6
DIQMTQSPSSLSASVGDRVTITCKASQDVSIGVAWYQQKPGKAPKLLIYSASYRYTGVPSRFSGSGSGTDFTLTISSLQPEDFATYYCQQYYITPYTFGQGTKVEIK

SEQ ID NO: 7 - protein, 214 aa, Artificial Sequence
  LC1
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 214 aa, Artificial Sequence
  LC2
DIQMTQSPSSLSASVGDRVTITCRASQDVNIAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 214 aa, Artificial Sequence
  LC3
DIQMTQSPSSLSASVGDRVTITCRASQDVNTAVAWYQQKPGKAPKLLIYSASFLYSGVPSRFSGSRSGTDFTLTISSLQPEDFATYYCQQHYTYPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 214 aa, Artificial Sequence
  LC4
DIQMTQSPSSLSASVGDRVTITCRASQDVNIAVAWYQQKPGKAPKLLIYSASFLYSGVPSRFSGSRSGTDFTLTISSLQPEDFATYYCQQHYTYPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 214 aa, Artificial Sequence
  LC5
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 214 aa, Artificial Sequence
  LC6
DIQMTQSPSSLSASVGDRVTITCKASQDVSIGVAWYQQKPGKAPKLLIYSASYRYTGVPSRFSGSGSGTDFTLTISSLQPEDFATYYCQQYYITPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 119 aa, Artificial Sequence [sequence omitted]

SEQ ID NO: 14 - protein, 120 aa, Artificial Sequence [sequence omitted]

SEQ ID NO: 15 - protein, 449 aa, Artificial Sequence
  HC1
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 16 - protein, 450 aa, Artificial Sequence
  HC2
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 17 - protein, 450 aa, Artificial Sequence
  HC3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 449 aa, Artificial Sequence
  HC4
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 19 - protein, 330 aa, Artificial Sequence
  FC1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 20 - protein, 330 aa, Artificial Sequence
  FC2
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 21 - protein, 471 aa, Artificial Sequence
  FC3
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 227 aa, Artificial Sequence
  FC4
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 23 - protein, 227 aa, Artificial Sequence
  FC5
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 24 - protein, 471 aa, Artificial Sequence
  FC6
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFKLYSKLTVDKSRWQQGNVFSCSVMHEALHNHYTQKSLSLSPGK

SEQ ID NO: 25 - protein, 471 aa, Artificial Sequence
  FC7
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSELTVDKSRWQQGNVFSCSVMHEALHNHYTQKSLSLSPGK

SEQ ID NO: 26 - protein, 471 aa, Artificial Sequence
  FC8
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 27 - protein, 471 aa, Artificial Sequence
  FC9
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPEVCTLPPSRDELTENQVSLWCLVKGFYPSDIAVEWESNGQPENNYKTTPPVLDSDGSFKLYSKLTVDKSRWQQGNVFSCSVMHEALHNHYTQKSLSLSPGK

SEQ ID NO: 28 - protein, 227 aa, Artificial Sequence
  FC10
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 29 - protein, 471 aa, Artificial Sequence
  FC11
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPRVCTLPPSRDELTKNQVSLWCLVKGFYPSDIAVEWESNGQPENNYKTTPPVLDSDGSFKLYSKLTVDKSRWQQGNVFSCSVMHEALHNHYTQKSLSLSPGK

SEQ ID NO: 30 - protein, 227 aa, Artificial Sequence
  FC12
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSRCELTENQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 31 - protein, 471 aa, Artificial Sequence
  FC13
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPEVCTLPPSRDELTENQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 32 - protein, 227 aa, Artificial Sequence
  FC14
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRCELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 33 - protein, 471 aa, Artificial Sequence
  FC15
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPRVCTLPPSRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 34 - protein, 227 aa, Artificial Sequence
  FC16
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSRCELTENQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 35 - protein, 471 aa, Artificial Sequence
  FC17
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCEVKGFYPSDIAVEWESNGQPENNYKTTPPVLDSDGSFFLYSALTVDKSRWQQGNVFSCSVMHEALHNHYTQKSLSLSPGK

SEQ ID NO: 36 - protein, 471 aa, Artificial Sequence
  FC18
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYDTTPPVLDSDGSFFLYSALTVDKSRWQQGNVFSCSVMHEALHNHYTQKSLSLSPGK

SEQ ID NO: 37 - protein, 227 aa, Artificial Sequence
  FC19
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLSSDGSFKLVSKLTVDKSRWQQGNVFSCSVMHEALHNHYTQKSLSLSPGK

SEQ ID NO: 38 - protein, 471 aa, Artificial Sequence
  FC20
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 39 - protein, 227 aa, Artificial Sequence
  FC21
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCGVKGFYPSDIAVEWESNGQPENNYKTTPPVLDSDGSFKLASKLTVDKSRWQQGNVFSCSVMHEALHNHYTQKSLSLSPGK

SEQ ID NO: 40 - protein, 227 aa, Artificial Sequence
  FC22
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDALTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 41 - protein, 471 aa, Artificial Sequence
  FC23
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVDTLPPDRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 42 - protein, 471 aa, Artificial Sequence
  FC24
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFKLYSKLTVDKSRWQQGNVFSCSVMHEALHNHYTQKSLSLSPGK

SEQ ID NO: 43 - protein, 227 aa, Artificial Sequence
  FC25
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSALTVDKSRWQQGNVFSCSVMHEALHNHYTQKSLSLSPGK

SEQ ID NO: 44 - protein, 471 aa, Artificial Sequence
  FC26
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLSSDGSFKLYSKLTVDKSRWQQGNVFSCSVMHEALHNHYTQKSLSLSPGK

SEQ ID NO: 45 - protein, 227 aa, Artificial Sequence
  FC27
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYDTTPPVLDSDGSFFLVSALTVDKSRWQQGNVFSCSVMHEALHNHYTQKSLSLSPGK

SEQ ID NO: 46 - protein, 227 aa, Artificial Sequence
  FC28
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCGVKGFYPSDIAVEWESNGQPENNYKTTPPVLDSDGSFFLASALTVDKSRWQQGNVFSCSVMHEALHNHYTQKSLSLSPGK

SEQ ID NO: 47 - protein, 471 aa, Artificial Sequence
  FC29
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVDTLPPSRDELTKNQVSLWCLVKGFYPSDIAVEWESNGQPENNYKTTPPVLDSDGSFKLYSKLTVDKSRWQQGNVFSCSVMHEALHNHYTQKSLSLSPGK

SEQ ID NO: 48 - protein, 227 aa, Artificial Sequence
  FC30
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDALTKNQVSLSCAVKGFYPSDIAVEWESNGQPENNYKTTPPVLDSDGSFFLVSALTVDKSRWQQGNVFSCSVMHEALHNHYTQKSLSLSPGK

SEQ ID NO: 49 - protein, 471 aa, Artificial Sequence
  FC31
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVDTLPPDRDELTKNQVSLWCLVKGFYPSDIAVEWESNGQPENNYKTTPPVLDSDGSFKLYSKLTVDKSRWQQGNVFSCSVMHEALHNHYTQKSLSLSPGK

SEQ ID NO: 50 - protein, 330 aa, Homo sapiens [sequence omitted]

SEQ ID NO: 51 - protein, 227 aa, Artificial Sequence
  Fc 1-1
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 52 - protein, 227 aa, Artificial Sequence
  Fc 2-1
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

(19) United States
(12) Patent Application Publication
(10) Pub. No.: US 2023/0151116 A1
(43) Pub. Date: May 18, 2023

(54) EFFICACIOUS DOSE FOR HER2 BISPECIFIC ANTIBODY

(71) Applicant: JIANGSU ALPHAMAB BIOPHARMACEUTICALS CO., LTD., Suzhou, Jiangsu (CN)

(72) Inventors: Ting XU, Suzhou, Jiangsu (CN); Junfang XU, Suzhou, Jiangsu (CN); Jing YANG, Suzhou, Jiangsu (CN); Jiazhu FANG, Suzhou, Jiangsu (CN); Pilin WANG, Suzhou, Jiangsu (CN); Ting CHEN, Suzhou, Jiangsu (CN)

(21) Appl. No.: 17/914,969

(22) PCT Filed: Mar. 26, 2021
(86) PCT No.: PCT/CN2021/083308

§ 371 Date: Sep. 27, 2022

(30) Foreign Application Priority Data
Mar. 27, 2020 (CN) PCT/CN2020/081727

(51) Int. Cl. C07K 16/32; A61P 35/00

(52) U.S. Cl. CPC C07K 16/32; A61P 35/00; C07K 2317/31

(57) ABSTRACT

The present application provides a method of treating tumor, comprising: administrating a dose of 15 mg/kg to 35 mg/kg of a HER2 bispecific antibody, which comprises a first and a second light chain, a first and a second heavy chain, and variable region of the light chain comprises an amino acid sequence as set forth in any one of SEQ ID NO: 1-6.

### BACKGROUND OF THE INVENTION

HER2 protein is a type I transmembrane growth factor receptor tyrosine kinase. It mediates signal transduction pathways that involve in cell proliferation, apoptosis regulation, and biological functions such as angiogenesis and lymphangiogenesis. HER2 positivity accounts for about 15-20% of breast cancers. Nevertheless, patient inevitably experienced progressive disease that urges new drug development.

Clinical translation of bispecific antibody could be challenging due to altered target engagement and difference between preclinical and clinical tumors. Development of population pharmacokinetics (PK)-tumor growth models within the modeling framework aid in understanding of the link between drug exposure, pharmacodynamics, tumor response and provide a tool for optimizing clinical dose selection.

And it is urgent and necessary to explore optimize dose for an the HER2 bispecific antibody in humans.

### SUMMARY OF THE INVENTION

The present application provides a method of treating breast cancer or inhibiting breast tumor growth in a subject in need of, and the method comprises administrating to the subject a dose of 15 mg/kg to 35 mg/kg of a bispecific antibody. And the present application provides an formulation, as well as a drug delivery device for use in.

In an aspect, the present application provides a method of preventing, alleviating or treating tumor or inhibiting tumor growth in a subject, comprising: administrating to the subject a dose of about 15 mg/kg to about 35 mg/kg of a HER2 bispecific antibody, wherein the HER2 bispecific antibody comprises a first light chain, a second light chain, a first heavy chain and a second heavy chain, wherein the first light chain and the second light chain is capable of assembling with a heavy chain of Pertuzumab and a heavy chain of Trastuzumab, respectively; wherein variable region of the first light chain and/or the second light chain comprises an amino acid sequence as set forth in any one of SEQ ID NO: 1-6.

In some embodiments, the variable region of the first light chain and/or the second light chain comprises an amino acid sequence as set forth in SEQ ID NO: 1. In some embodiments, the first light chain and the second light chain is selected from a light chain of Pertuzumab or a mutant thereof, a light chain of Trastuzumab or a mutant thereof, respectively.

In some embodiments, the first light chain comprises an amino acid sequence as set forth in any one of SEQ ID NO: 7-12, and/or, the second light chain comprises an amino acid sequence as set forth in any one of SEQ ID NO: 7-12.

In some embodiments, the heavy chain variable regions are a heavy chain variable region of Pertuzumab and a heavy chain variable region of Trastuzumab, respectively. In some embodiments, the variable region of the first heavy chain comprises an amino acid sequence as set forth in SEQ ID NO: 13; and variable region of the second heavy chain comprises an amino acid sequence as set forth in SEQ ID NO: 14.

In some embodiments, the first heavy chain and the second heavy chain comprises a constant region, and the constant region is originated from human IgG constant region.
In some embodiments, the Fc fragment sequences of the first heavy chain or the second heavy chain comprise sequences as set forth in any one of SEQ ID NO: 19-49, 51-52.

In some embodiments, the first heavy chain or the second heavy chain comprises a sequence as set forth in any one of SEQ ID NO: 15-18.

In some embodiments, the dose is about 20 mg/kg to about 30 mg/kg.

In some embodiments, the dose is about 20 mg/kg.

In some embodiments, the dose is about 30 mg/kg.

In some embodiments, the HER2 bispecific antibody is administrated once every two weeks or once every three weeks.

In some embodiments, the dose is about 20 mg/kg, and the HER2 bispecific antibody is administered once every two weeks.

In some embodiments, the dose is about 30 mg/kg, and the HER2 bispecific antibody is administered once every three weeks.

In some embodiments, the subject was not responsive to a conventional therapy for HER2-related tumor.

In some embodiments, the conventional therapy for HER2-related tumor comprises administrating HER2-ADC, MBC hormone, Taxane, pyrotinib, neratinib, tucatinib, trastuzumab and/or pertuzumab.

In some embodiments, the conventional therapy for HER2-related tumor comprises administrating docetaxel, capecitabine and/or lapatinib.

In some embodiments, the tumor comprises solid tumor.

In some embodiments, the tumor comprises metastatic tumor, early tumor and/or locally advanced tumor.

In some embodiments, the tumor comprises HER2 positive tumor and/or HER2 low-expression tumor.

In some embodiments, the tumor comprises a breast cancer and/or a gastric cancer.

In some embodiments, the breast cancer comprises HER2 positive breast cancer and/or HER2 low-expression breast cancer.

In some embodiments, the breast cancer comprises early breast cancer, locally advanced breast cancer and/or metastatic breast cancer; and/or the gastric cancer comprises early gastric cancer, locally advanced gastric cancer and/or metastatic gastric cancer.

In some embodiments, the HER2 bispecific antibody is administrated by intravenous administration.

In another aspect, the present application provides a formulation for use in preventing, alleviating or treating tumor or inhibiting tumor growth in a subject in need of, the formulation comprises at least 5 μg/mL of a HER2 bispecific antibody, wherein the HER2 bispecific antibody comprises a first light chain, a second light chain, a first heavy chain and a second heavy chain, wherein the first light chain and the second light chain is capable of assembling with a heavy chain of Pertuzumab and a heavy chain of Trastuzumab, respectively; wherein variable region of the first light chain and/or the second light chain comprises an amino acid sequence as set forth in any one of SEQ ID NO: 1-6.

In some embodiments, in the formulation, the variable region of the first light chain and/or the second light chain comprises an amino acid sequence as set forth in SEQ ID NO: 1.

In some embodiments, in the formulation, first light chain and the second light chain is selected from a light chain of Pertuzumab or a mutant thereof, a light chain of Trastuzumab or a mutant thereof, respectively.

In some embodiments, in the formulation, the first light chain comprises an amino acid sequence as set forth in any one of SEQ ID NO: 7-12, and/or, the second light chain comprises an amino acid sequence as set forth in any one of SEQ ID NO: 7-12.

In some embodiments, in the formulation, the heavy chain variable regions are a heavy chain variable region of Pertuzumab and a heavy chain variable region of Trastuzumab, respectively.

In some embodiments, in the formulation, the variable region of the first heavy chain comprises an amino acid sequence as set forth in SEQ ID NO: 13; and variable region of the second heavy chain comprises an amino acid sequence as set forth in SEQ ID NO: 14.

In some embodiments, in the formulation, the first heavy chain and the second heavy chain comprises a constant region, and the constant region is originated from human IgG constant region.

In some embodiments, in the formulation, Fc fragment sequences of the heavy chains comprise sequences as set forth in any one of SEQ ID NO: 19-49, 51-52.

In some embodiments, in the formulation, the first heavy chain or the second heavy chain comprises a sequence as set forth in any one of SEQ ID NO: 15-18.

In some embodiments, the formulation comprises about at least 12 μg/mL of the bispecific antibody.

In some embodiments, the formulation comprises about at least 20 μg/mL of the bispecific antibody.

In some embodiments, the formulation is packaged in a container.

In another aspect, the present application provides a drug delivery device for use in preventing, alleviating or treating tumor or inhibiting tumor growth in a subject in need of, comprises a formulation comprising at least 5 μg/mL of a HER2 bispecific antibody, wherein the HER2 bispecific antibody comprises a first light chain, a second light chain, a first heavy chain and a second heavy chain, wherein the first light chain and the second light chain is capable of assembling with a heavy chain of Pertuzumab and a heavy chain of Trastuzumab, respectively; wherein variable region of the first light chain and/or the second light chain comprises an amino acid sequence as set forth in any one of SEQ ID NO: 1-6.

In some embodiments, in the drug delivery device, the variable region of the first light chain and/or the second light chain comprises an amino acid sequence as set forth in SEQ ID NO: 1.

In some embodiments, in the drug delivery device, the first light chain and the second light chain is selected from a light chain of Pertuzumab or a mutant thereof, a light chain of Trastuzumab or a mutant thereof, respectively.

In some embodiments, in the drug delivery device, the first light chain comprises an amino acid sequence as set forth in any one of SEQ ID NO: 7-12, and/or, the second light chain comprises an amino acid sequence as set forth in any one of SEQ ID NO: 7-12.

In some embodiments, in the drug delivery device, the heavy chain variable regions are a heavy chain variable region of Pertuzumab and a heavy chain variable region of Trastuzumab, respectively.

In some embodiments, in the drug delivery device, the variable region of the first heavy chain comprises an amino acid sequence as set forth in SEQ ID NO: 13; and variable region of the second heavy chain comprises an amino acid sequence as set forth in SEQ ID NO: 14.

In some embodiments, in the drug delivery device, the first heavy chain and the second heavy chain comprises a constant region, and the constant region is originated from human IgG constant region.

In some embodiments, in the drug delivery device, the Fc fragment sequences of the heavy chains comprise sequences as set forth in any one of SEQ ID NO: 19-49, 51-52.

In some embodiments, in the drug delivery device, the first heavy chain or the second heavy chain comprises a sequence as set forth in any one of SEQ ID NO: 15-18.

In some embodiments, the formulation comprises about at least 12 μg/mL of the bispecific antibody.

In some embodiments, wherein the formulation comprises about at least 20 μg/mL of the bispecific antibody.

In some embodiments, wherein the formulation is packaged in a container.

Additional aspects and advantages of the present application will become readily apparent to those skilled in this art from the following detailed description, wherein only illustrative embodiments of the present application are shown and described. As will be realized, the present application is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1 illustrates tumor volume dynamics in xenograft models by study and dose level of said HER2 bispecific antibody in present application.

FIG. 2 illustrates Goodness-of-fit (GOF) of tumor inhibition model for NCI-N87 and Calu-3 xenograft models.

FIG. 3A-3D illustrates predicted tumor size dynamics in human under different exposures of said HER2 bispecific antibody in present application by the translational model.

FIG. 4A illustrates dose-normalized said HER2 bispecific antibody in present application concentration within dosing Cycle 1.

FIG. 4B illustrates Goodness-of-fit plots for the population PK model.

FIG. 5 illustrates comparison of predicted SLD changes from baseline across different dosing regimens.

FIG. 6 illustrates structural model used to build said HER2 bispecific antibody in present application exposure-SLD relationship in humans.

FIG. 7 illustrates SLD data used in interim ER analysis.

FIG. 8 illustrates tumor growth inhibition (TGI) studies used to establish preclinical PK-PD for said HER2 bispecific antibody in present application.

FIG. 9 illustrates tumor growth change from baseline by xenograft models by study.

FIG. 10 illustrates tumor growth change from baseline by dosing level of said HER2 bispecific antibody in present application.

FIG. 11 illustrates tumor growth change from baseline by dosing regimen of said HER2 bispecific antibody in present application.

FIG. 12 illustrates target concentration of said HER2 bispecific antibody in present application in mice dependent on tumor volume.

FIG. 13 illustrates comparison of Ctrough concentration among candidate dosing regimens.

FIG. 14 illustrates the responses and durations of response in evaluable patients shown in Swimmer plot by dose level.

FIGS. 15A-15B illustrate the tumor responses of all evaluable patients shown in waterfall plot and spider plot by dose level.

FIGS. 16A-16F illustrate pretreatment and posttreatment scans from a patient with relapsed breast cancer who had a partial response.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

In the present application, the term “HER2” as used herein, generally refers to the type I transmembrane protein, also known as c-erbB2, ErbB2 or Neu, belonging to the family of epidermal growth factor receptors. In the context of the present application, the term “HER2” also encompasses homologues, variants and isoforms, including splice isoforms, of HER2. The term “HER2” further encompasses proteins having the sequence of one or more of a HER2 homologue, variant and isoform, as well as fragments of the sequences, provided that the variant proteins (including isoforms), homologous proteins and/or fragments are recognized by one or more HER2 specific antibodies, such as provided as Pertuzumab, Trastuzumab and Margetuximab. The HER2 may be a human HER2. The human HER2 gene is mapped to chromosomal location 17q12, and the genomic sequence of HER2 gene can be found in GenBank at NG_007503.1. In human, there are five HER2 isoforms: A, B, C, D, and E; the term “HER2” is used herein to refer collectively to all HER2 isoforms.

In the present application, the term “antibody” as used herein, generally refers to an immunoglobulin or a fragment or a derivative thereof, and encompasses any polypeptide comprising an antigen-binding site, regardless whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)2, Fv, scFv, Fd, dAb, and other antibody fragments that retain antigen-binding function, i.e., the ability to bind, for example, HER2 specifically. Typically, such fragments would comprise an antigen-binding domain.

In the present application, the term “bispecific antibody” refers to an antibody that can respectively bind with two different antigens or the antigen epitopes thereof. For example, the bispecific antibody may comprise at least one kind of light chain or the fragment thereof, as well as at least one kind of heavy chain or the fragment thereof. For example, the bispecific antibody may comprise one light chain or the fragment thereof, which may specifically bind to both a first antigen or the antigen epitope thereof and a second antigen or the antigen epitope thereof. For example, the bispecific antibody may comprise two heavy chains or the fragment thereof, which bind to the first antigen or the antigen epitope thereof and the second antigen or the antigen epitope thereof, respectively. For example, the first antigen or the antigen epitope thereof and the second antigen or the antigen epitope thereof may be two different HER2 antigen.

In the present application, the term “HER2-positive” or “HER2-positive” as used herein, generally refers to a tumor comprising cells which have HER2 protein present at their cell surface. HER2 protein may be overexpressed, e.g., by gene amplification. the solid tumor overexpressing HER2 may be rated by immunohistochemical scores according to the number of copies of HER2 molecules expressed per cell, and can been determined biochemically (refers to Hudziak et al., Proc. Natl. Acad. Sci. USA 84: 7159-7163 [1987]). For example, the HER2-positive solid tumor may comprise a HER-2 positive breast cancer. the HER-2 positive breast cancer may test positive for estrogen receptor and may be a HER2 nonamplified invasive breast cancer. the HER2-positive breast cancer may be advanced. the HER2-positive breast cancer may be metastatic.

In the present application, the term “HER2 low-expression” refers to a tumor comprising cells which expresses very low level of HER2. HER2 low-expression may refer to HER2-negative tumors that test IHC 1⁺ or 2⁺ and FISH⁻. The expression level of HER2 may be measured by immunohistochemistry or FISH. For example, the group with low levels of HER2 may be more likely to be of higher grade, EGFR-positive and ER/HER3/HER4-negative.

In the present application, the term “solid tumor” refers to an abnormal mass of tissue that usually does not contain liquid areas. the solid tumor may be malignant, and may belong to cancer. Different types of solid tumors are named for the type of cells that form them. For example, the solid tumor may comprise breast cancer.

In the present application, the term “metastatic” refers to a tumor that spreads from its site of origin to another part of the body. For many types of tumor, it may be also called stage IV (4) tumor. The metastatic tumor may develop when the tumor cells break away from the main tumor and enter the bloodstream or lymphatic system. For example, breast cancer that spreads to the lung may be called metastatic breast cancer.

In the present application, the term “early tumor” refers to a tumor that has not grown deeply into nearby tissues. The early tumor may be called early-stage cancer, and/or may be called stage I (1) tumor. The early tumor may have not been spread to distant regions. In the present application, the term “locally advanced tumor” refers to a tumor having grown outside the body part it started in but has not yet spread to other parts of the body. For example, locally advanced breast cancer may be a subset of breast cancer characterized by the most advanced breast tumors in the absence of distant metastasis.

In the present application, the term “treating” as used herein, generally refers to clinical intervention in an attempt to alter the natural course of the individual being treated, and may be performed either for prophylaxis or during the course of clinical pathology. Desirable effects of treatment may also comprise preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. For example, the HER2 bispecific antibody may be used to delay development of a disease or to slow the progression of a disease.

In the present application, the term “preventing” as used herein, generally refers to delaying the onset, hindering the progress, hindering the appearance, protection against, inhibiting or eliminating the emergence, or reducing the incidence, of such damages, effects or symptoms of a disease or disorder.

In the present application, the term “alleviating” as used herein, generally refers to a process by which the severity of a sign or symptom of a disorder is decreased. the alleviating may comprise alleviating but not eliminating a sign or symptom of a disease or disorder.

In the present application, the term “subject” as used herein, generally refers to an animal, for example, a human. For example, the subject may comprise “non-human animals”, which may comprise mammals such as rats, mice, rabbits, sheep, cats, dogs, cows, pigs, and non-human primates.

In the present application, the term “conventional therapy for HER2-related tumor” as used herein, generally refers to administrating any substances or drugs which block the growth of HER2-related tumor. The conventional therapy for HER2-related tumor may interfere the function of specific molecules responsible for HER2-related (for example, HER2-positive and/or HER2 low expression) tumor cell proliferation and survival. The conventional therapy for HER2-related tumor may comprise any approved drugs specific for treating HER2-related tumor (for example, the HER2-related tumor may be a solid tumor, for example, the HER2-related tumor may at any stage). The conventional therapy for HER2-related tumor may comprise a first-line and/or a second-line approved drug for treating HER2-related tumor (for example, may have been approved for treating a HER2-positive breast cancer). The conventional therapy for HER2-related tumor may comprise any approved drugs suitable for treating HER2-related tumor, including the drugs for universal tumor treatment, for example, a chemotherapy.

In the present application, the term “trastuzumab” as used herein, generally refers to a whole human HER2 monoclonal antibody used to treat breast cancer and stomach cancer. Its brand name can be Herceptin, Herzuma or Ogivri. Trastuzumab may be specifically used for cancer that is HER2 receptor positive.

In the present application, the term “MBC hormone” as used herein, generally refers to hormone therapy for treating breast cancer. In some embodiments, said hormone therapy may stop hormones (for example estrogen, or progesterone) from attaching to the receptors in breast cancer cells. For example, said hormone therapy may comprise administrating Tamoxifen and/or Toremifene.

In the present application, the term “Taxane” as used herein, generally refers to a class of diterpenes. The Taxane may also be used to treat metastatic breast cancer. The CAS number of Taxane may be 1605-68-1. Taxane may have the following formula:

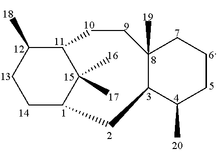

In the present application, the term “HER2-ADC” as used herein, generally refers to a HER2 targeting antibody drug conjugate and is capable of binding to HER2 on the surface of tumor cell. For example, the HER2-ADC may comprise a trastuzumab emtansine (T-DM1), which may be indicated for treatment of HER2-positive metastatic breast cancer. For example, the HER2-ADC may comprise a Trastuzumab deruxtecan (DS-8201a), which may be indicated for treatment of adult patients with unresectable or metastatic HER2-positive breast cancer. For example, the HER2-ADC may comprise a SYD985, in which trastuzumab is linked to the duocarmycin prodrug seco-duocarmycin-hydroxybenzamide-azaindole orseco-DUBA via a cleavable linker.

In the present application, the term “pyrotinib” as used herein, generally refers to an irreversible dual pan-ErbB receptor tyrosine kinase inhibitor. The pyrotinib may target EGFR, HER2, and HER4. The pyrotinib may be used for the treatment of HER2-positive advanced solid tumours. Pyrotinib Racemate is the racemate of Pyrotinib, and Pyrotinib Racemate is a compound having the following formula:

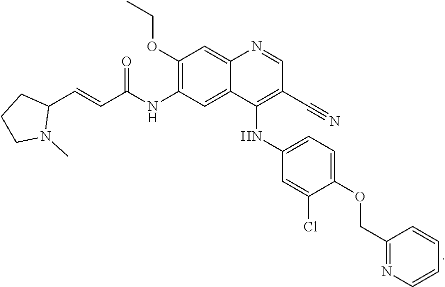 [SMILES: CCOc1cc2ncc(C#N)c(Nc3ccc(OCc4ccccn4)c(Cl)c3)c2cc1NC(=O)/C=C/C1CCCN1C]

In the present application, the term “neratinib” as used herein, generally refers to a tyrosine kinase inhibitor. Neratinib may be used for extended adjuvant treatment of adults with early stage hormone receptor positive HER2-overexpressed/amplified breast cancer. Neratinib is a compound having the following formula:

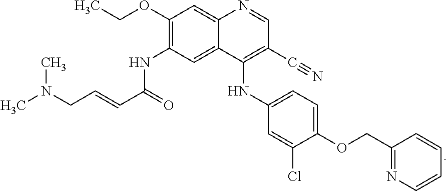 [SMILES: CCOc1cc2ncc(C#N)c(Nc3ccc(OCc4ccccn4)c(Cl)c3)c2cc1NC(=O)/C=C/CN(C)C]

In the present application, the term “tucatinib” as used herein, generally refers to a small molecule inhibitor of HER2. Tucatinib may be used for advanced unresectable or metastatic HER2-positive breast cancer. Tucatinib is a compound having the following formula:

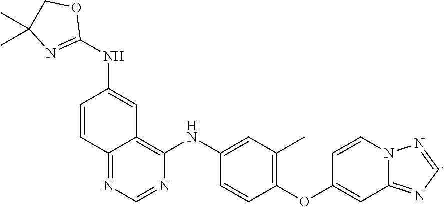 [SMILES: Cc1cc(Nc2ncnc3ccc(NC4=NC(C)(C)CO4)cc23)ccc1Oc1ccn2ncnc2c1]

In the present application, the term “pertuzumab” as used herein, generally refers to a monoclonal antibody used for treating HER2-positive breast cancer. The amino acid sequences of the variable light and variable heavy chains of the pertuzumab (OMNITARG®) may be referred to WO2006033700A2.

In the present application, the term “trastuzumab” as used herein, generally refers to a monoclonal antibody that interferes with the HER2/neu receptor (tradenames Herclon, Herceptin) (Hudis, 2007, N. Engl. J. Med. 3577(1):39-51).

In the present application, the term “docetaxel” as used herein, generally refers to the active ingredient of TAXOTERE® or else TAXOTERE® itself. Docetaxel is a compound having the following formula:

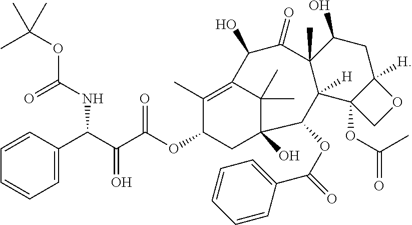

In the present application, the term “capecitabine” as used herein, generally refers to a chemotherapeutic agent that is a prodrug that is converted into 5-FU in the tissues. The chemical name of the capecitabine is pentyl [1-(3,4-dihydroxy-5-methyltetrahydrofuran-2-yl)-5-fluoro-2-oxo-1H-pyrimidin-4-yl]carbamates.

In the present application, the term “lapatinib” as used herein, generally refers to an orally active drug for breast cancer and other solid tumors. It is a dual tyrosine kinase inhibitor which interrupts the HER2/neu and epidermal growth factor receptor (EGFR) pathways. It acts as a dual reversible TKI for both these receptors, thus blocking the downstream MAPK/Erk1/2 and PI3K/AKT pathways. Lapatinib is a compound having the following formula:

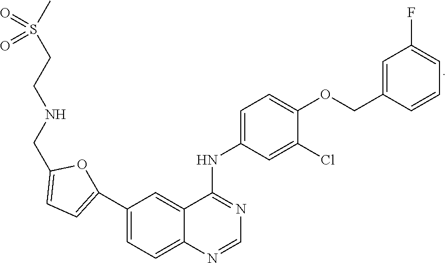 [SMILES: CS(=O)(=O)CCNCc1ccc(-c2ccc3ncnc(Nc4ccc(OCc5cccc(F)c5)c(Cl)c4)c3c2)o1]

In the present application, the term “formulation” as used herein, generally refers to a composition that comprise the HER2 bispecific antibody of the present application. For example, said formulation may further comprise one or more pharmaceutically acceptable excipients. For example, said pharmaceutically acceptable excipients may comprise the one recorded in Fourth Edition, Royal Pharmaceutical Society of Great Britain, Science & Practice Publishers; or Remingtons: The Science and Practice of Pharmacy (Nineteenth Edition, Mack Publishing Company).

In the present application, the term “drug delivery device” as used herein, generally refers to a device that comprises the formulation in present application. In the present application, the drug delivery device may deliver the HER2 bispecific antibody to the site of the tumor and/or the site in need in a subject.

In the present application, the term “a” as used herein, generally not means to limit as a singular. In certain embodiments, the term “a” may refer to a plural form. As used throughout this disclosure, the singular forms “a,” “an,” and “the” include plural reference unless the context clearly dictates otherwise.

In the present application, the term “about” as used herein, generally refers to a variation that is within a range of normal tolerance in the art, and generally means within ±10%, such as within 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

In an aspect, the present application provides a method of preventing, alleviating or treating tumor or inhibiting tumor growth in a subject, comprising: administrating to the subject a dose of about 15 mg/kg to about 35 mg/kg of a HER2 bispecific antibody, wherein the HER2 bispecific antibody comprises a first light chain, a second light chain, a first heavy chain and a second heavy chain, wherein the first light chain and the second light chain is capable of assembling with a heavy chain of Pertuzumab and a heavy chain of Trastuzumab, respectively; wherein variable region of the first light chain and/or the second light chain comprises an amino acid sequence as set forth in any one of SEQ ID NO: 1-6.

For example, the HER2 bispecific antibody may be a bispecific antibody or the antigen binding portion thereof, and the bispecific antibody or the antigen binding portion thereof may have a first light chain and a second light chain, and the first light chain and the second light chain may comprise a same amino acid sequence. For example, as the first light chain and the second light chain comprises the same amino acid sequence, the bispecific antibody or the antigen binding portion thereof may have a common light chain.

For example, the common light chain may be obtained via engineering from two different original monoclonal antibodies which may capable of binding to different epitopes of human HER2, respectively. In some cases, the common light chain may be originated from the light chain of either of the two original monoclonal antibodies. In some cases, the common light chain may be modified on the basis of the light chain of either of the two original monoclonal antibodies.

For example, the modification may comprise an insertion, a deletion and/or a substitution in at least one amino acid positions of an amino acid sequence of the light chain of either of the two original monoclonal antibodies. In some cases, the purpose of the modification is to maintain the affinity between the bispecific antibody or the antigen binding portion thereof to the corresponding epitopes.

In present application, light chain constant regions of the bispecific antibody or the antigen binding portion thereof may be of κ type or λ, type; the κ-type light chain constant region may comprise various allotypes, such as Km1, Km2 and Km3; the λ-type light chain constant region may comprise various allotypes, such as CL1, CL2, CL3, CL6 and CL7.

In present application, the HER2 bispecific antibody may be a bispecific antibody or the antigen binding portion thereof, and the bispecific antibody or the antigen binding portion thereof may have a first heavy chain and a second heavy chain.

In present application, the first heavy chain and the second heavy chain are capable of correctly assembling with the light chains respectively under physiological conditions or during in vitro protein expression.

For example, the first light chain and the second light chain may be capable of assembling with a heavy chain of Pertuzumab and a heavy chain of Trastuzumab, respectively. For example, variable region of the first light chain and/or the second light chain may comprise an amino acid sequence as set forth in SEQ ID NO: 1.

For example, the first light chain and the second light chain may be selected from a light chain of Pertuzumab or a mutant thereof, a light chain of Trastuzumab or a mutant thereof, respectively.

For example, variable region of the first light chain and variable region of the second light chain may be the variable region of light chain of Trastuzumab. For example, the first light chain and the second light chain may be the light chain of Trastuzumab. For example, the first light chain and the second light chain may comprise an amino acid sequence as set forth in any one of SEQ ID NO: 7-12. For example, the first light chain and the second light chain may comprise an amino acid sequence as set forth in SEQ ID NO: 7.

For example, variable region of the first heavy chain may be a heavy chain variable region of Pertuzumab, and variable region of the second heavy chain may be a heavy chain variable region of Trastuzumab. For example, variable region of the first heavy chain may comprise an amino acid sequence as set forth in SEQ ID NO: 13; and variable region of the second heavy chain may comprise an amino acid sequence as set forth in SEQ ID NO: 14.

In the present application, the first heavy chain and/or the second heavy chain may comprise a constant region. For example, the constant region may be originated from human IgG constant region. For example, heavy chain constant region of the first heavy chain and heavy chain constant region of the second heavy chain may be identical to or different from each other. In some cases, the amino acid sequences of the variable region and the CH1 domain of the first heavy chain and the second heavy chain are identical to those of the original monoclonal antibodies.

In the present application, the bispecific antibody or the antigen binding portion thereof may block both ligand-dependent and ligand-independent HER2 signaling pathway. For example, the IgG1 Fc fragment of the bispecific antibody or the antigen binding portion thereof may bind to FcRγIIIa and may mediate potent ADCC effect. For example, the bispecific antibody or the antigen binding portion thereof may enhance a HER2 internalization and/or may show better anti-tumor activity in preclinical models than using the original monoclonal antibodies alone, e.g. trastuzumab and pertuzumab.

In some cases, the light chain constant region and/or the heavy chain constant region of the bispecific antibody or the antigen binding portion thereof may comprise a modification in order to obtain a better ADCC, CDC, endocytosis, stability, immunogenicity and/or half-life; furthermore, the modification may also facilitate formation of the heterodimer protein during antibody expression. In the present application, technologies for modifying an Fc fragment of the heavy chain are known in the art.

For example, Fc fragment of the first heavy chain may comprise an amino acid sequence as set forth in any one of SEQ ID NO: 19-49, 51-52; and Fc fragment of the second heavy chain may comprise an amino acid sequence as set forth in SEQ ID NO: 19-49, 51-52. For example, Fc fragment of the first heavy chain may comprise an amino acid sequence as set forth in SEQ ID NO: 19; and Fc fragment of the second heavy chain may comprise an amino acid sequence as set forth in SEQ ID NO: 20.

For example, Fc fragment of the first heavy chain may comprise an amino acid sequence as set forth in SEQ ID NO: 51; and Fc fragment of the second heavy chain may comprise an amino acid sequence as set forth in SEQ ID NO: 52.

For example, the first heavy chain may comprise an amino acid sequence as set forth in SEQ ID NO: 17; and the second heavy chain may comprise an amino acid sequence as set forth in SEQ ID NO: 18.

For example, the first heavy chain may comprise an amino acid sequence as set forth in SEQ ID NO: 15; and the second heavy chain may comprise an amino acid sequence as set forth in SEQ ID NO: 16.

In present application, the HER2 bispecific antibody may comprise a first light chain, a second light chain, a first heavy chain and a second heavy chain, variable region of the first light chain and/or the second light chain may comprise a sequence as set forth in SEQ ID NO: 1; the variable region of the first heavy chain may comprise an amino acid sequence as set forth in SEQ ID NO: 13; and variable region of the second heavy chain may comprise an amino acid sequence as set forth in SEQ ID NO: 14. The first heavy chain may comprise an amino acid sequence as set forth in SEQ ID NO: 15; and the second heavy chain may comprise an amino acid sequence as set forth in SEQ ID NO: 16.

The amino acid sequence in the present application may also comprise an amino acid sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100%) identity to an amino acid sequence as set forth in any one of SEQ ID NO: 1-52 in the sequence listing. For example, the amino acid sequence in the present application may comprise an amino acid sequence having one or more (e.g., 1-2, 1-3, 1-4, 1-5, 1-6, 1-7, 1-8, 1-9, 1-10, or more) amino acid deletion, insertion and/or substitution in the amino acid sequence as set forth in any one of SEQ ID NO: 1-52 in the sequence listing.

In the present application, the dose may be about 20 mg/kg to about 30 mg/kg (for example, a dose of at least about 20 mg/kg, a dose of at least about 20.5 mg/kg, a dose of at least about 21 mg/kg, a dose of at least about 21.5 mg/kg, a dose of at least about 22 mg/kg, a dose of at least about 22.5 mg/kg, a dose of at least about 23 mg/kg, a dose of at least about 23.5 mg/kg, a dose of at least about 24 mg/kg, a dose of at least about 24.5 mg/kg, a dose of at least about 25 mg/kg, a dose of at least about 25.5 mg/kg, a dose of at least about 26 mg/kg, a dose of at least about 26.5 mg/kg, a dose of at least about 27 mg/kg, a dose of at least about 27.5 mg/kg, a dose of at least about 28 mg/kg, a dose of at least about 28.5 mg/kg, a dose of at least about 29 mg/kg, a dose of at least about 29.5 mg/kg and a dose of at least about 30 mg/kg). For example, the dose may be about 20 mg/kg. For example, the dose may be about 30 mg/kg.

In the present application, the HER2 bispecific antibody may be administrated once every two weeks or once every three weeks. For example, the dose may be about 20 mg/kg, and the HER2 bispecific antibody may be administered once every two weeks. For example, the dose may be 30 mg/kg, and the HER2 bispecific antibody may be administered once every three weeks.

For example, the subject may be not responsive to a conventional therapy for HER2-related tumor. For example, the conventional therapy for HER2-related tumor may comprise administrating drugs which is specific for targeting HER2. For example, may comprise HER2 antigen binding protein (for example, anti-HER2 antibody), the conjugate thereof and/or HER2 specific inhibitors. For example, the conventional therapy for HER2-related tumor may comprise administrating HER2-ADC, MBC hormone, Taxane, pyrotinib, neratinib, tucatinib, trastuzumab and/or pertuzumab. For example, the conventional therapy for HER2-related tumor may comprise administrating drugs which is universal for treating tumor. For example, may comprise any available chemotherapy drugs. For example, the conventional therapy for HER2-related tumor may comprise administrating docetaxel, capecitabine and/or lapatinib.

In present application, the not responsive may refer to a syndrome of the tumor of the subject has not been alleviated significantly after administrated with the conventional therapy for HER2-related tumor. For example, the syndrome may comprise a decrease of the volume of a tumor. For example, the syndrome may comprise an extension of the OS, ORR and/or PFS.

In the present application, the subject in need of may have been failed in a conventional therapy for HER2-related tumor, and the conventional therapy for HER2-related tumor may comprise administrating trastuzumab, MBC hormone and/or Taxane.

For example, the subject in need of may have failed in a conventional therapy for HER2-related tumor. For example, the conventional therapy for HER2-related tumor may comprise administrating Trastuzumab, HER2 TKI and HER2 ADC. For example, the median number of prior lines of the conventional HER2 target therapy among the subject in need of may be 2 (range: 1-12).

For example, the subject in need thereof may have HER2-positive metastatic breast cancer whose disease has progressed after treatment with trastuzumab and/or a taxane.

For example, the subject in need thereof may have been treated with prior hormonal treatment. For example, the hormonal treatment may comprise administrating drugs blocking estrogen receptors. For example, the hormonal treatment may comprise treatment with Tamoxifen and/or Toremifene. For example, the taxane may comprise Paclitaxel (Taxol) and docetaxel (Taxotere).

For example, the subject in need thereof may have been treated with the hormonal treatment first, and then been treated with trastuzumab and/or a taxane. For another example, the subject in need thereof may have been treated with trastuzumab and/or a taxane first, and then been treated with the hormonal treatment.

In the present application, the tumor may comprise a solid tumor. For example, the tumor may comprise metastatic tumor, early tumor and/or locally advanced tumor. For example, the tumor may comprise HER2 positive tumor and/or HER2 low-expression tumor.

For example, the tumor may comprise a breast cancer and/or a gastric cancer. For example, the breast cancer may comprise HER2 positive breast cancer and/or HER2 low-expression breast cancer. For example, the breast cancer may comprise early breast cancer, locally advanced breast cancer and/or metastatic breast cancer. For example, the gastric cancer may comprise early gastric cancer, locally advanced gastric cancer and/or metastatic gastric cancer. For example, the subject in need thereof may have histologically or cytologically proven diagnosis of HER2-positive adenocarcinoma of the breast at the time of diagnosing locally advanced unresectable or metastatic disease.

For example, the breast cancer may be HR negative (HR⁻) or HR positive (HR⁺) breast cancer. For example, the cancer may be HR⁻HER2-positive positive breast cancer. For another example, the cancer may be HR⁺HER2-positive positive breast cancer.

In present application, the subject in need thereof may be treated with the HER2 bispecific antibody at scheduled regimen until progressive disease, unacceptable toxicity or withdrawal of informed consent whichever comes first.

In present application, the treatment may result in a disease response. For example, the disease response may comprise a decrease of the tumor volume. For example, Tumor assessment according to RECIST 1.1 criteria was performed at baseline, every 8 weeks for QW and Q2W schedule and every 6 weeks for Q3W schedule within 12 months and every 12 weeks thereafter.

The HER2 bispecific antibody of present application may be administered by the same route of administration or by different routes of administration. For example, the HER2 bispecific antibody may be administrated by intravenous administration.

For example, the HER2 bispecific antibody may be administrated as a 90-minutes intravenous infusion for the initial dose. For example, the HER2 bispecific antibody may be administrated shorted to a 60-minutes intravenous infusion for subsequent doses.

In present application, a cycle may be defined as 28 days for Q2W (once for two weeks) dosing and 21 days for Q3W (once for three weeks) dosing.

In present application, the formulation may comprise at least about 5 μg/mL (for example, at least about 5 μg/mL, at least about 5.5 μg/mL, at least about 6 μg/mL, at least about 6.5 μg/mL, at least about 7 μg/mL, at least about 7.5 μg/mL, at least about 8 μg/mL, at least about 8.5 μg/mL, at least about 9 μg/mL, at least about 9.5 μg/mL, at least about 10 μg/mL, at least about 10.5 μg/mL, at least about 11 μg/mL, at least about 11.5 μg/mL, at least about 12 μg/mL, at least about 12.5 μg/mL, at least about 13 μg/mL, at least about 13.5 μg/mL, at least about 14 μg/mL, at least about 14.5 μg/mL, at least about 15 μg/mL, at least about 15.5 μg/mL, at least about 16 μg/mL, at least about 17 μg/mL, at least about 16.5 μg/mL, at least about 17 μg/mL, at least about 17.5 μg/mL, at least about 18 μg/mL, at least about 18.5 μg/mL, at least about 19 μg/mL, at least about 19.5 μg/mL, at least about 20 μg/mL, at least about 27 μg/mL, at least about 78 μg/mL or more) of the HER2 bispecific antibody. For example, the formulation may comprise at least about 5 μg/mL of the bispecific antibody. For example, the formulation may comprise at least about 20 μg/mL of the bispecific antibody.

For example, the formulation may be packaged in a container. For example, the device may be a container. In present application, the container may be a “container”, a pen or a syringe. For example, the container may be a prefilled container, a prefilled pen, or a prefilled syringe. For example, the intravenous administration is from a saline container. For example, the container may be connected to a channel comprising a tube and/or a needle.

In present application, the formulation may be a liquid formulation. For example, the formulation may be prepared in an aqueous carrier. For example, a stabilizer may be added in an amount no greater than that which may result in a viscosity undesirable or unsuitable for intravenous administration. For example, the liquid formulation may also include one or more of a buffering agent, a surfactant, and a preservative.

For example, the formulation may be administrated by intravenous administration.

### Examples

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., s or sec, second(s); min, minute(s); h or hr, hour(s); i.m., intramuscular(ly); i.p., intraperitoneal(ly); s.c., subcutaneous(ly); and the like.

### Materials

HER2 bispecific antibody: The HER2 bispecific antibody has a common light chain and the first heavy chain and the second heavy chain, wherein the common light chain comprises an amino acid sequence as set forth in SEQ ID No: 7; the first heavy chain comprises an amino acid sequence as set forth in SEQ ID No: 15 and the second heavy chain comprises an amino acid sequence as set forth in SEQ ID No: 16.

Human cancer cell lines: Calu-3 (human lung cancer cell line) and NCI-N87 (human gastric cell line) were purchased from Cell Resource Center of Shanghai Institutes for Biological Sciences, Chinese Academy of Sciences (Shanghai, China). The cell lines were characterized by the vendor; no further cell line authentication was conducted.

Preclinical Xenograft models: HER2 bispecific antibody antitumor activity was evaluated using NCI-N87 and Calu-3 xenograft models. In these experiments, BALB/c mice were subcutaneously injected in the right flank with 4˜6×10⁶ NCI-N87 or Calu-3 tumor cells. Treatment of HER2 bispecific antibody started 8 days after tumor cell implantation with initial tumor ranging from 100 to 150 mm3. BALB/c mice (n=5-6 per group) received weekly or bi-weekly intraperitoneal (i.p.) injections of either PBS, HER2 bispecific antibody for 4-5 weeks. Tumor size was determined with a caliper twice weekly and tumor volume was calculated using the equation: tumor volume (mm³)=(width²×length)×0.5.

Mice were euthanized on Day 28˜35 or when tumor size reached 2000 mm³. Before euthanization, one time-point blood was drawn from each animal subject for the measurement of plasma HER2 bispecific antibody concentration. A total of 40 animal subjects from placebo group and 50 animal subjects from HER2 bispecific antibody treatment groups were included in the analysis, including 780 tumor volume observations (246 observations from placebo group and 534 observations from HER2 bispecific antibody groups).

Mouse xenograft studies were performed by Suzhou Alphamab, China and the experimental protocol design was compliant to the rules of 3 Rs for Animal Welfare.

Software: Datasets assembly was performed using SAS® (version 9.3). Graphical data exploration, model-based simulations, and additional data processing were performed using R (version 3.5.1). Nonlinear mixed effects modeling was performed using NONMEM (version 7.4, ICON Development Solutions, Ellicott City, Md., USA). All models were estimated using first order conditional estimation method with η-ε interaction (FOCE-I).

### Clinical Trials

### Study Design and Patient Selection

This phase I, multicenter-center, open-label, 3+3, dose-escalation study was designed to evaluate the safety, tolerability, pharmacokinetics (PK), and preliminary antitumor activity, and identify the recommended phase II dose (RP2D) of the HER2 bispecific antibody in patients with HER2-positive MBC. The study protocol was approved by institutional review boards before patient recruitment and conducted in accordance with International Conference on Harmonization E6 Guidelines for Good Clinical Practice. Each patient provided signed informed consent before study enrollment.

Eligible patients were 18-75 years old and had histologically confirmed HER2 positive metastatic breast cancer. HER2 positivity status was determined according to ASCO/CAP 2018 guideline. Patients had received at least 1 prior line of anti-HER2 therapy in the metastatic setting, at least one measurable disease per RECIST 1.1 and baseline left-ventricular ejection fraction (LVEF)≥55%. Patients were excluded if they had unstable brain metastasis, malignant meningitis, had history of symptomatic interstitial lung disease, history of cumulative doxorubicin dose exceeding 300 mg/m² or equivalent or medically significant cardiac disorder. If patients had next-generation sequencing results from blood or archival tumor samples before informed consent was signed, the information would be collected.

In the 3+3, dose-escalation stage, patients received the HER2 bispecific antibody treatments with dosing consisted of 5 mg/kg QW, 10 mg/kg QW, 20 mg/kg Q2W, and 30 mg/kg Q3W. Eligible patients were given the HER2 bispecific antibody intravenously dose on the 21-day or 28-day cycle until disease progression, unacceptable toxicity, withdrawal of informed consent. Three patients were initially assigned to a starting dose level of 5 mg/kg. A dose-limiting toxicity (DLT) was defined as a toxic reaction related to study treatment after the administration that is unacceptable due to severity and/or irreversibility and limits further dose escalation. The DLT evaluation period was 28 days for dosing frequency of QW and Q2W, and 21 days for Q3W. Once an objective response (partial response or complete response) was observed at a certain dose level, the dose level will be expanded to enroll additional 23 to 25 patients to explore the efficacy, safety and tolerability of the HER2 bispecific antibody. The highest dose level at which no more than one of six patients had a DLT was considered the maximum tolerated dose (MTD). Population pharmacokinetics and pharmacodynamics approach was used to determine RP2D if MTD cannot be identified.

### Safety Assessment

Safety evaluations were conducted at every cycle, including all adverse events (AE), DLT (for dose-escalation stage), clinical laboratory parameters, electrocardiogram (ECG), ECOG performance status, vital signs, and physical examinations. AEs were assessed according to the NCI CTCAE, version 4.03 and were monitored until 90 days after the last dose.

### Clinical Activity

Tumor imaging assessments were conducted every 6 weeks during first 12 months and every 12 weeks thereafter per investigator review according to Response Evaluation Criteria in Solid Tumors (RECIST) guidelines version 1.1 until progressive disease, starting a new anti-tumor therapy or withdrawal of informed consent. Patients who achieved an objective response was confirmed at least 4 weeks apart. Baseline tissue and peripheral blood ctDNA second-generation sequencing (NGS) information from 22 patients who had the information before the informed consent of this study was collected, and the correlation between NGS and efficacy was analyzed.

### PK Analyses

Samples of the HER2 bispecific antibody serum concentration were collected on Day 1 of Cycles 1 and 2 at pre-dose, 30-minutes after infusion, end-of-infusion (EOI), 2, 6, 24, 72 and 168 hours after EOI, and Days 1 and 15 of Cycles 3, 4, 5, and 6 at pre-dose and EOI (1 cycle=28 days for Q2W dosing and 21 days for Q3W dosing). Samples were shipped frozen on dry ice and stored under −80° C. until analysis. The concentrations of the HER2 bispecific antibody in Human serum were measured by a quantitative sandwich Electrochem-iluminescent (ECL) assay. MSD Quickplex120 was used for data collection and Watson LIMS was used for data processing. Datasets assembly was performed using SAS® (version 9.3). Graphical data exploration, model-based simulations, and additional data processing were performed using R (version 3.5.1). Non compartment analysis was performed using Phoenix WinNonlin (Pharsight, Mountain View, Calif.) version 8.0. Nonlinear mixed effects modeling was performed using NONMEM (version 7.4, ICON Development Solutions, Ellicott City, Md., USA).

### Sample Size Determination and Statistical Analysis

The size of the dose-escalation cohort was based on a 3+3 phase I trial design. The sample size of the expansion cohorts was calculated using a one-sided accurate test method, with a presumption that each cohort in the dose expansion phase would enroll approximately 60 subjects. Assuming that the ORR in patients with HER2-positive MBC who failed the prior anti-HER2 treatments was 8-10%, a size of 60 patients can provide a power of 80% to detect an increase in ORR at a level of 30% (α=0.05). The safety analyses were based on the safety analysis set, efficacy analyses were based on the full analysis set (FAS) of efficacy.

Demographics, baseline characteristics, AEs, laboratory toxicities, and DLT were summarized using descriptive statistics. ORR, DCR and CBR were reported as point estimates and 95% exact binomial CIs using Clopper Pearson method. Survival outcomes including PFS and OS were estimated by the Kaplan-Meier method. For all PK endpoints, descriptive statistics and graphical display were conducted. The Tmax was described with median, 25 and 75 percentiles, minimum and maximum values. Statistical computation was performed with SAS 9.4.

### Immunogenicity Assessments

The clinical immunogenicity strategy followed a tiered approach consistent with industry practices for biotherapeutics (FDA, Guidance for Industry: Immunogenicity Assessment for Therapeutic Protein Products (Silver Spring, Md., August 2014)). First, samples were screened in a validated in-solution bridging ELISA to detect potential positive responses to the different domains in the bispecific antibody. Secondly, samples with positive signals were confirmed in the same ELISA after a competitive binding step with the bispecific antibody. Then, confirmed positive samples were analyzed for ADA domain specificity by competitive binding with the bispecific antibody component. Lastly, relative levels of ADA were determined by titer.

### Statistical Analysis

The safety analyses were based on the safety analysis set, efficacy analyses were based on the full analysis set (FAS) of efficacy. The safety analysis set included patients who had received at least one dose of study treatment. FAS included patients who had completed at least one dose of study treatment and had been evaluated for tumor response. Pharmacokinetic analysis population included patients with evaluable PK data. Depending on the availability of data, the analysis of different PK parameters may include different numbers of patients.

The primary endpoint of the dose-escalation phase was to DLT in the first cycle of treatment. The primary endpoint of the dose-expansion phase was investigator assessed objective response rate (ORR) per RECIST 1.1, defined as the proportion of patients who have confirmed complete or partial response. Secondary endpoints were duration of response (DOR), defined as time from first documented CR or PR to disease progression or death due to any cause, progression-free survival time (PFS), defined as time from first trial treatment to disease progression or death due to any cause, clinical benefit rate (CBR) was defined as the percentage of patients with CR, PR and SD≥24 weeks. Safety assessments included the types, incidence and severity of the treatment related adverse events (TRAE); abnormal laboratory tests; PK parameters which included but were not limited to the total area under the concentration-time curve (AUC0-t), the peak plasma concentration (Cmax), the elimination half-life (T_{1/2}).

### Example 1 Clinical Study

Clinical Study Design: The first-in-human (FIH) clinical trial (NCT03619681, protocol approved by the Ethics Committees of all participating clinical sites) is an ongoing Phase 1 study of HER2 bispecific antibody in HER2 expressing breast cancer, gastric/gastroesophageal junction cancer and other locally advanced/metastatic solid tumors. All patients have provided written informed consent before study entry. All subjects enrolled were HER2-positive breast cancer patients who have failed available HER2 target therapies, including at least trastuzumab. The median number of prior lines of HER2 target therapies among the patients was 2 (range: 1-12). Patients in dose-escalation were treated with doses ranging from 5 mg/kg QW to 30 mg/kg Q3W. Patients in dose-expansion were treated with either 20 mg/kg Q2W and 30 mg/kg Q3W. HER2 bispecific antibody was administered as a 90-minutes intravenous infusion for the initial dose and shorted to a 60-minutes intravenous infusion for subsequent doses. Samples of HER2 bispecific antibody serum concentration were collected on Day 1 of Cycles 1 and 2 at pre-dose, 30-minutes after infusion, end-of-infusion (EOI), 2, 6, 24, 72 and 168 hours after EOI, and Days 1 and 15 of Cycles 3, 4, 5, and 6 at pre-dose and EOI (1 cycle=28 days for Q2W dosing and 21 days for Q3W dosing). Samples were shipped frozen on dry ice and stored under −80° C. until analysis. Subjects were treated with HER2 bispecific antibody at scheduled regimen until progressive disease, unacceptable toxicity or withdrawal of informed consent whichever comes first. Tumor assessment according to RECIST 1.1 criteria was performed at baseline, every 8 weeks for QW and Q2W schedule and every 6 weeks for Q3W schedule within 12 months and every 12 weeks thereafter. Sum of longitudinal diameters of target lesions determined by RECIST 1.1 criteria are used to build tumor growth model in humans.

### Example 2 Inhibiting Tumor Growth

Translational tumor growth inhibition modeling: Longitudinal tumor volume data including 780 observations from 87 mice in NCI-N87 and Calu-3 xenograft models were used to describe the relationship between HER2 bispecific antibody trough concentration and tumor volume dynamics. A tumor growth inhibition model, accounting for both natural tumor growth in the xenograft models and tumor killing rate by HER2 bispecific antibody, was developed. Standard model evaluations were performed to qualify the tumor growth inhibition model in mice.

To further bridge the anti-tumor effect of HER2 bispecific antibody to human exposures, a translational tumor model was next extrapolated from the developed tumor growth inhibition model in mice. Simulations were next run to explore different scenarios of initial tumor volumes and tumor doubling times in humans under various HER2 bispecific antibody concentrations.

### Tumor Growth Inhibition Data and Modeling

Tumor volume data from the mice xenograft models show a plausible HER2 bispecific antibody dose-response relationship (FIG. 1). Starting from the base structural model adopted from established preclinical tumor models, different forms of tumor growth component were tested. A saturable tumor growth component similar to was found to best describe the xenograft data, and an Eₘₐₓ drug effect model was found to describe HER2 bispecific antibody anti-tumor effect better than a linear relationship. The final tumor growth inhibition model to describe mice xenograft data is as follows:

$$\frac{dTV ( t )}{d t} = K G * ( 1 - \frac{TV ( t )}{TG 50 + TV ( t )} ) * TV ( t ) - KD * \frac{Conc}{KC 50 + Conc} * TV ( t )$$

Where KG is the maximal natural tumor growth rate, and KD is maximal tumor killing rate associated with the maximal HER2 bispecific antibody effect. TV(t) is the tumor volume at time t. Conc is the trough concentration of HER2 bispecific antibody. TG50 is the tumor volume at which the tumor growth rate decreases to 50% of the maximal rate, and KC50 is the HER2 bispecific antibody trough concentration level at which the tumor killing rate decreases to 50% of the maximal tumor killing effect of HER2 bispecific antibody.

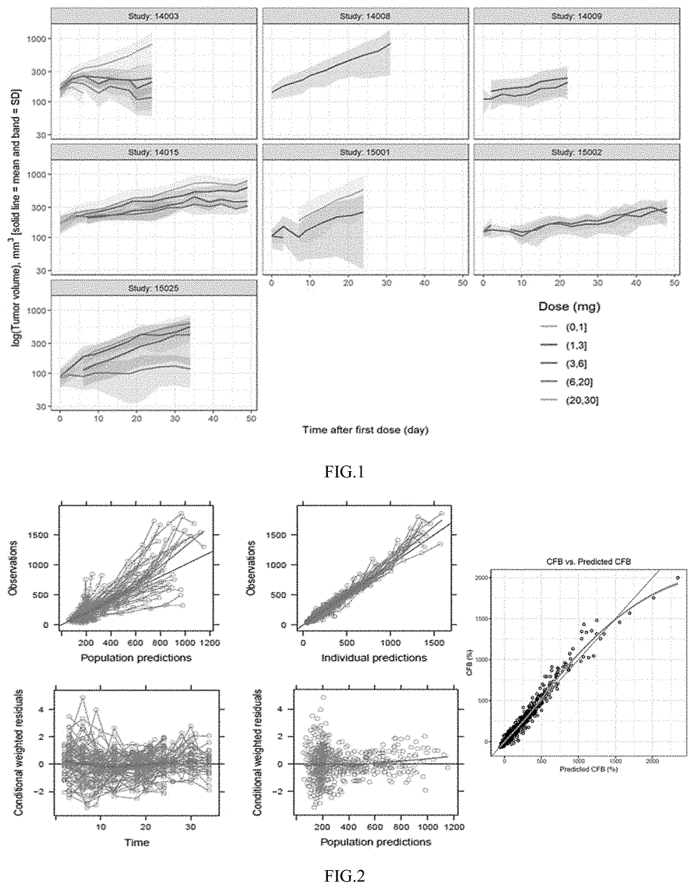

The estimated tumor growth model parameters are provided in Table 1. Goodness-of-fit plots demonstrate adequate fit and minimal bias of the model (FIG. 2).

TABLE 1
Parameter estimates for tumor growth inhibition model in mice

| | | RSE, |
|---|---|---|
| | Estimates | % |
| Parameters | | |
| K_{G}: maximum tumor growth rate (1/Day) | 0.248 | 22.5 |
| K_{D}: maximum tumor death rate (1/Day) | 0.106 | 16.7 |
| TYPKG: NCI-H522 effect on KG | −0.616 | 7.3 |
| TG₅₀: Tumor volume with half of maximum | 157 | 37.7 |
| growth rate (mm³) | | |
| KC₅₀: HER2 bispecific antibody concentration | 2.27 | 67.4 |
| achieving half of maximum | | |
| killing rate (□g/mL) | | |
| Random effect | | |
| Var (ETA on KG) | 0.126 | 31.6 |
| Var (ETA on KD) | 0.257 | 63.8 |
| SD (proportional residual error) | 0.185 | 5.6 |

The final model indicates that tumor growth in mice slows down at higher tumor volume, and a higher concentration is needed to achieve the same tumor-growth-inhibition target when tumor volume is lower. Specifically, HER2 bispecific antibody trough concentration of 78.1 μg/mL is needed to achieve 95% tumor growth inhibition when the tumor volume in the mice xenograft model is 200 mm³ (Table 2).

TABLE 2
HER2 bispecific antibody trough concentrations to
achieve tumor stasis or 95% tumor growth inhibition
at different tumor volumes in the xenograft models

| Tumor | HER2 bispecific antibody | HER2 bispecific antibody |
|---|---|---|
| volume | Ctrough to achieve | Ctrough to achieve |
| (mm³) | tumor stasis (μg/mL) | 95% TGI (μg/mL) |
| 200 | NA | 78.1 |
| 220 | 67.6 | 27.8 |
| 240 | 27.1 | 16.9 |
| 260 | 17 | 12.1 |
| 280 | 12.3 | 9.46 |
| 300 | 9.7 | 7.75 |

### Translational Tumor Model for HER2 Bispecific Antibody Human Projection

A literature-documented tumor growth equation more relevant to the observed tumor growth dynamics in breast cancer patients was used to replace the tumor growth component in the developed tumor growth inhibition model for mice. The translational tumor model for HER2 bispecific antibody human projection is as follows:

$$\frac{dTV ( t )}{d t} = \frac{{λ}_{0} ⨯ ( 1 - \frac{T V ( t )}{{V}_{max}} )}{{( 1 + {( \frac{{λ}_{0}}{{λ}_{1}} ⨯ TV ( t ) )}^{ψ} )}^{\frac{1}{ψ}}} ⨯ TV ( t ) - KD ⨯ \frac{Conc}{{KC}_{50} + Conc} ⨯ TV ( t )$$

Key parameters for tumor growth in breast cancer patients were decided from literature documented values. In particular, λ₀ represents the growth parameter from exponential growth phase of the tumor, and was calculated from a tumor doubling time during the exponential growth phase of 25 days (CV %=200%). λ₁ represents the growth parameter from linear growth phase of the tumor, and was calculated from a tumor doubling time of 621 days (CV %=85%) associated with the linear growth phase. Maximum achievable tumor volume Vₘₐₓ was set to 523.8 cm³ based on maximal achievable tumor radius of 5 cm. Ψ is fixed at 20 to reflect the empirical shape parameter switching between exponential and linear growth. Lastly, initial tumor volume TV(0)=2745.5 mm³ was calculated from an initial tumor lesion length of 19 mm (range 7-70 mm) and initial lesion breadth of 17 mm (range 7-80 mm).

On the other hand, tumor killing component and model parameters intrinsic to HER2 bispecific antibody effect in tumor-killing, i.e. K_{D} and KC₅₀, were assumed constant across species and remained the same as estimated from the tumor growth inhibition model in mice: K_{D}=0.106/day and KC₅₀=2.57 μg/mL.

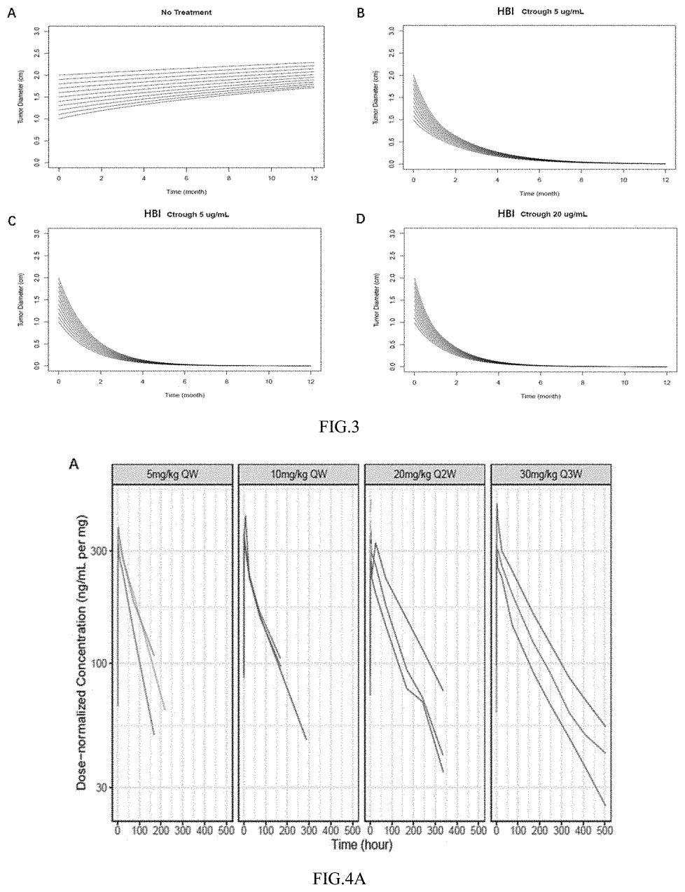

Simulations were next conducted to predict tumor size dynamics in humans under different HER2 bispecific antibody trough concentration levels or without treatment, in order to project efficacious HER2 bispecific antibody exposure levels in humans (FIG. 3A-3D). In FIG. 3, A represents No treatment, exponential tumor doubling time 25 day; B represents trough HER2 bispecific antibody concentration 5 μg/mL, exponential tumor doubling time 25 day; C represents trough HER2 bispecific antibody concentration 5 μg/mL, exponential tumor doubling time 250 day and D represents trough HER2 bispecific antibody concentration 20 μg/mL, exponential tumor doubling time 25 day.

The simulation results from the translational tumor growth inhibition model showed that tumor stasis can be achieved at HER2 bispecific antibody trough concentrations lower than 20 μg/mL, and more aggressive tumors (i.e. an exponential doubling time of 25 days in comparison to a less aggressive growth with doubling time of 250 days) will take longer time to achieve tumor stasis under a given concentration. While a trough concentration of 20 μg/mL can noticeably reduce the time to model stasis than a trough concentration of 5 μg/mL, there seems to be very limited gain in efficacy when HER2 bispecific antibody trough concentration goes higher than 20 μg/mL.

### Example 3 PK Measurement

Population PK modeling: HER2 bispecific antibody concentration data including 324 PK observations from 20 patients in the FIE study and nonlinear mixed-effects modeling approach were used in the population PK analysis. A two-compartment model with linear elimination was established to describe HER2 bispecific antibody PK profile. Potential covariate effects were evaluated on relevant PK parameters. Inter-individual variability was considered for all PK parameters, including central and peripheral volumes, clearance, and inter-compartmental clearance, as follows:

Pᵢ=Pₚₒₚ×exp(ηᵢ) where Pᵢ is the individual parameter estimate for individual i, and Pₚₒₚ is the typical population parameter estimate, and where ηᵢ was assumed to be distributed normally with mean 0 and variance ω̅ ². Residual variability was described by both a proportional and an addictive component:

C_{obs,i,j}=C_{pred,ij}×(1+ε_{p,ij})+ε_{a,ij}, where C_{obs,ij} represents the observed concentration for individual i and observation j, C_{prod,ij} represents the individual predicted concentration, ε_{p,ij} the proportional error and ε_{a,ij} the addictive error, following normal distributions N ˜(0, σ²) with different σ². Standard model evaluations were performed to qualify population PK model for HER2 bispecific antibody.

To further determine exposure levels under different candidate dosing regimens of HER2 bispecific antibody, simulations were performed for 7 dosing scenarios (without loading: 5 mg/kg QW, 10 mg/kg QW, 20 mg/kg Q2W, 20 mg/kg Q3W; with loading in the first cycle: 20 mg/kg Q2W with 20 mg/kg QW loading on Days 1 and 8, 30 mg/kg Q3W with 20 mg/kg QW loading on Days 1 and 8 and 30 mg/kg Q2W with 30 mg/kg QW loading on Days 1 and 8), each for 30 weeks of dosing time on 1000 simulated individual patients. For each simulated patient, individual PK parameters were sampled from the distributions estimated by the population PK analysis. Individual body weights (the only covariate in the population PK model) were sampled from a log-normal distribution with mean and standard deviation calculated from the PK analysis dataset. The median and 90% predictive intervals of HER2 bispecific antibody trough, maximum and average concentrations were later summarized for each dosing scenario and compared across all scenarios.

### Population PK Analysis in Breast Cancer Patients

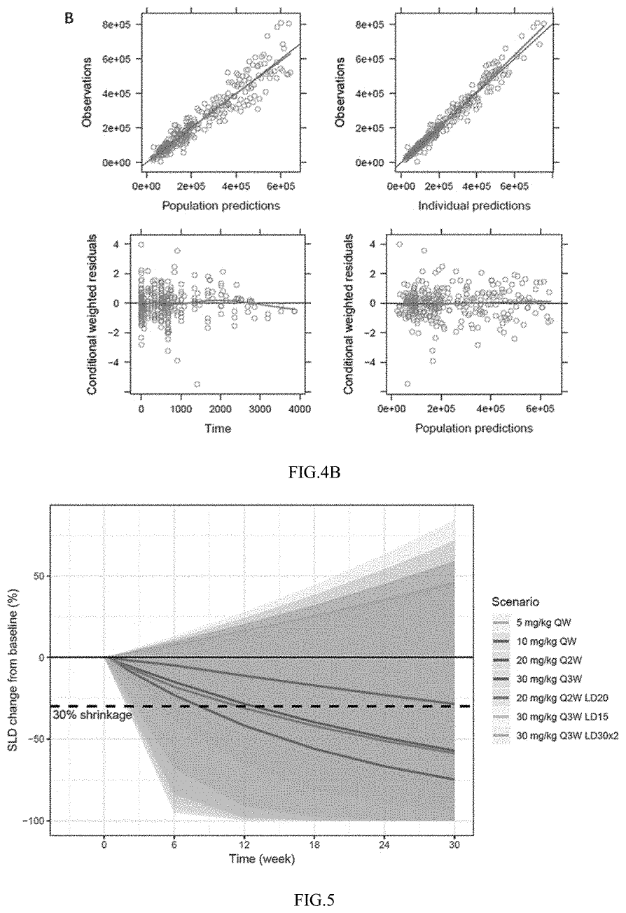

For population PK analysis, 324 post-dose HER2 bispecific antibody serum concentration observations from 20 patients were available and included in the analysis. Baseline demographics and characteristics for these patients were summarized in Table 3. Overall, the concentration data show clear characteristics of two-compartment disposition and the exposures are proportional to dose within the tested dose range (FIG. 4A).

TABLE 3
Summary of patient demographics and baseline characteristics in population analysis dataset

| | Total | 10 mg/kg QW | 20 mg/kg Q2W | 30 mg/kg Q3W | 5 mg/kg QW |
|---|---|---|---|---|---|
| | (N = 20) | (N = 3) | (N = 11) | (N = 3) | (N = 3) |
| Age (yr) | | | | | |
| Median (min, | 54.0 (32.0, 68.0) | 57.0 (43.0, 57.0) | 49.0 (32.0, 68.0) | 56.0 (41.0, 62.0) | 56.0 (45.0, 57.0) |
| max) | | | | | |
| Mean (sd) | 51.2 (9.8) | 52.3 (8.1) | 50.1 (11.5) | 53.0 (10.8) | 52.7 (6.7) |
| Albumin (g/L) | | | | | |
| Median (min, | 45.7 (38.0, 49.3) | 48.9 (42.6, 49.1) | 45.4 (38.0, 48.3) | 44.7 (41.0, 46.8) | 46.4 (42.9, 49.3) |
| max) | | | | | |
| Mean (sd) | 45.1 (3.3) | 46.9 (3.7) | 44.5 (3.4) | 44.2 (2.9) | 46.2 (3.2) |
| ALT (U/L) | | | | | |
| Median (min, | 13.6 (7.8, 41.0) | 14.0 (9.0, 17.7) | 13.3 (7.8, 41.0) | 12.3 (11.7, 14.8) | 20.7 (8.1, 36.2) |
| max) | | | | | |
| Mean (sd) | 18.1 (9.9) | 13.6 (4.4) | 19.8 (11.0) | 12.9 (1.6) | 21.7 (14.1) |
| AST (U/L) | | | | | |
| Median (min, | 25.6 (15.1, 56.0) | 26.2 (24.2, 28.1) | 26.9 (15.1, 56.0) | 22.2 (18.0, 23.0) | 25.3 (20.5, 50.0) |
| max) | | | | | |
| Mean (sd) | 27.1 (10.4) | 26.2 (2.0) | 27.8 (11.5) | 21.1 (2.7) | 31.9 (15.8) |
| CRCL (mL/min) | | | | | |
| Median (min, | 97.8 (58.5, 155.6) | 92.0 (58.5, 134.9) | 98.6 (61.1, 127.3) | 99.5 (91.7, 103.4) | 72.4 (65.4, 155.6) |
| max) | | | | | |
| Mean (sd) | 96.3 (26.2) | 95.1 (38.3) | 95.7 (22.2) | 98.2 (5.9) | 97.8 (50.2) |
| SCR (umol/L) | | | | | |
| Median (min, | 58.0 (38.0, 91.0) | 62.0 (52.0, 91.0) | 58.0 (45.0, 76.0) | 49.0 (44.0, 58.0) | 67.0 (38.0, 73.0) |
| max) | | | | | |
| Mean (sd) | 59.6 (12.3) | 68.3 (20.3) | 59.9 (8.8) | 50.3 (7.1) | 59.3 (18.7) |
| SLDB (mm) | | | | | |
| Median (min, | 46.8 (10.0, 181.9) | 114.4 (45.9, 181.9) | 37.0 (10.0, 154.7) | 43.9 (19.7, 48.6) | 62.5 (17.9, 64.3) |
| max) | | | | | |
| Mean (sd) | 55.5 (46.1) | 114.1 (68.0) | 46.5 (41.2) | 37.4 (15.5) | 48.2 (26.3) |
| Total | | | | | |
| Bilirubin(umol/L | | | | | |
| Median | 8.6 (3.8, 15.1) | 15.0 (5.5, 15.1) | 6.8 (3.8, 11.8) | 10.8 (9.1, 14.5) | 8.3 (6.2, 13.0) |
| (min, max) | | | | | |
| Mean (sd) | 8.9 (3.6) | 11.9 (5.5) | 7.4 (2.7) | 11.5 (2.8) | 9.2 (3.5) |
| Weight (kg) | | | | | |
| Median | 58.4 (44.4, 77.5) | 65.5 (61.1, 68.9) | 59.0 (44.4, 77.5) | 53.5 (51.0, 57.7) | 55.0 (54.8, 59.3 |
| (min, max) | | | | | |
| Mean (sd) | 59.3 (9.0) | 65.2 (3.9) | 60.0 (11.2) | 54.1 (3.4) | 56.4 (2.5) |

A two-compartment model with linear clearance from the central compartment describes the data well (FIG. 4B). Body weight was found to be a significant covariate on both central volume and clearance. The parameter estimates of the final model were estimated with reasonable range and good precisions (Table 4).

TABLE 4
Parameter estimates for the population PK model

| Parameter | Estimate | RSE |
|---|---|---|
| Clearance: CL; L/d | 0.322 | 9.7% |
| Central volume: V1; L | 2.98 | 3.3% |
| Intercompartmental clearance: Q; L/d | 0.271 | 14.4% |
| Peripheral volume: V2; L | 3.27 | 34.6% |
| SD of proportional residual error | 0.108 | 13.7% |
| SD of additive residual error | 12600 | 27.5% |
| Body weight effect on central volume: | 0.474 | 41.4% |
| exponent | | |
| Body weight effect on clearance: | 1.49 | 30.1% |
| exponent | | |
| Variance of IIV on CL | 0.051 | 57.1% |
| Variance of IIV on V₁ | 0.0181 | 41.2% |
| Variance of IIV on Q | 0.0412 | 46.8% |
| Variance of IIV on V₂ | 0.402 | 33.6% |

Simulations based on the final population PK model for HER2 bispecific antibody were performed to predict steady state trough concentration at different dosing regimens. As described in previous, for each candidate dosing regimen, 1000 simulated subjects were sampled parametrically from the estimated distributions of between-subject variability on PK parameters. Body weights for the simulated subjects were sampled from a log-normal distribution with a mean of 58.7 kg in the linear scale and a standard deviation of 0.148 in the log scale, derived from the patients in the population PK analysis dataset. Percent of subjects with steady state trough concentration above the threshold of 20 μg/mL and steady state peak concentration above the threshold of 300 μg/mL for each simulated dosing regimen are provided in Table 5.

TABLE 5
Predicted percent of subjects with C_{min, ss}
and C_{max, ss} above given thresholds

| | Percent (%) with | Percent (%) with |
|---|---|---|
| Scenario | C_{min, ss} > 20 □g/mL | C_{max, ss} > 400 □g/mL |
| 5 mg/kg QW | 99.8 | 0 |
| 10 mg/kg QW | 100 | 20.1 |
| 20 mg/kg Q2W | 100 | 84.2 |
| 30 mg/kg Q3W | 99.8 | 100 |
| 20 mg/kg Q2W LD20 | 99.9 | 78.6 |
| 30 mg/kg Q3W LD20 | 98.7 | 97.9 |
| 30 mg/kg Q3W LD30 | 100 | 99.9 |

The results suggested that more than 98% of simulated subjects can achieve a trough concentration above 20 μg/mL at steady state. On the other hand, approximately 80% of simulated subjects can achieve a maximum concentration above 400 μg/mL with 20 mg/kg Q2W regimen and more than 95% of simulated subjects can achieve a maximum concentration above 400 μg/mL with 30 mg/kg Q3W regimen, but no simulated subjects in 5 mg/kg QW regimen, and only about 20% in 10 mg/kg QW regimen, can achieve this threshold.

### Example 4 Efficacy in Patients

Preliminary ER analysis of HER2 bispecific antibody efficacy in patients: A preliminary interim analysis on the relationship between HER2 bispecific antibody exposure and response of tumor size—represented by the sum of the longitudinal diameter of the target lesions, or SLD, was conducted when the first batch of SLD data including 66 observations from 24 patients who had at least one post-dose SLD observation became available. Among the 24 patients, 20 were included in the previously described population PK analysis, for whom the individual post-hoc PK parameters were used to derive their exposures to HER2 bispecific antibody. For the remaining 4 patients, point estimates of PK parameters from the population PK analysis and their individual body weight were used to derive their exposures. Due to the limitation of data availability, tumor growth rate constant was fixed to an empirical value of 0.0228 per week by assuming a growth of 20% in SLD within 8 weeks, namely if remain untreated, the study population will develop RECIST 1.1 criteria determined progressive disease at 6-8 weeks apart. HER2 bispecific antibody-induced tumor killing rate constant was estimated with inter-individual variability. Different exposure metrics (Cₘᵢₙ, Cₘₐₓ, and Cave at steady state) were tested in the ER analysis. After the ER model for SLD was developed and qualified, simulations of SLD time courses under different candidate dosing regiments were performed. For each dosing scenario, 100 subjects were sampled parametrically for their PK and ER parameters from the established population PK and interim ER models, and covariates were resampled with replacement from the 24 subjects in SLD dataset through bootstrap. Each subject was assumed to be treated by HER2 bispecific antibody for 30 weeks in total, and SLD values were calculated once every 6 weeks. After simulations, time courses of SLD percent change from baseline, non-progression (SLD change from baseline rate, and rate of tumor shrinkage from baseline, were summarized and compared across dosing scenarios.

### Interim ER Analysis of Human Efficacy Data

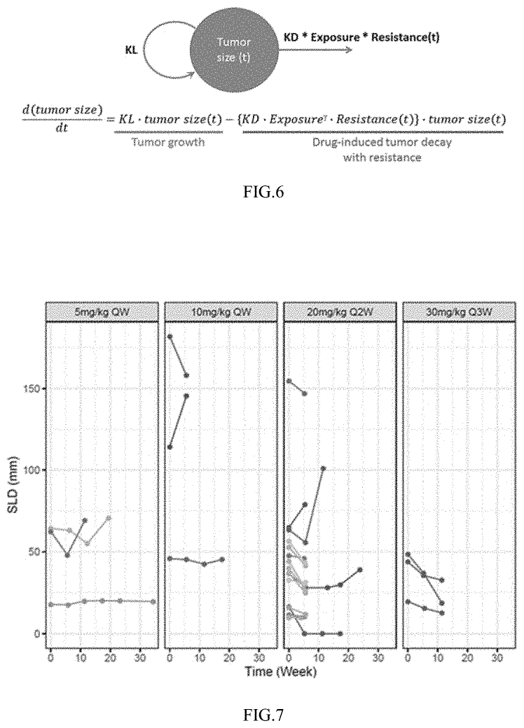

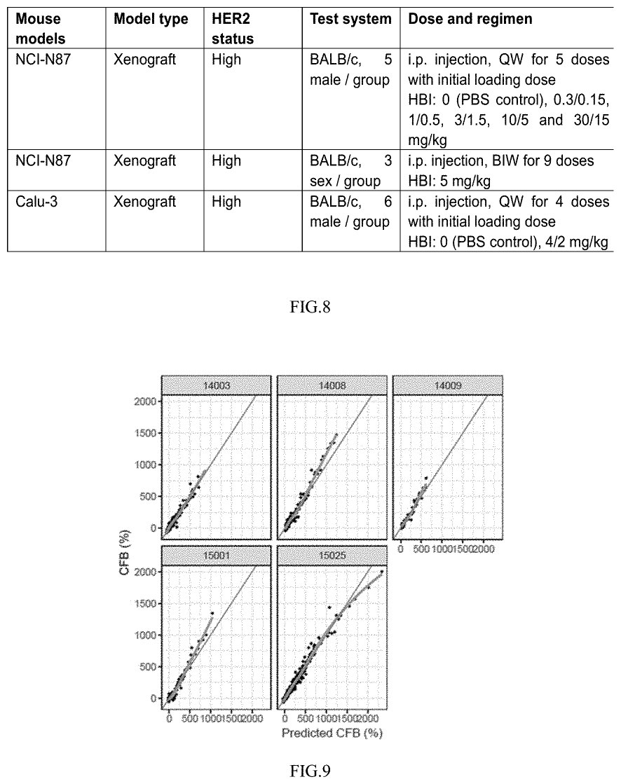

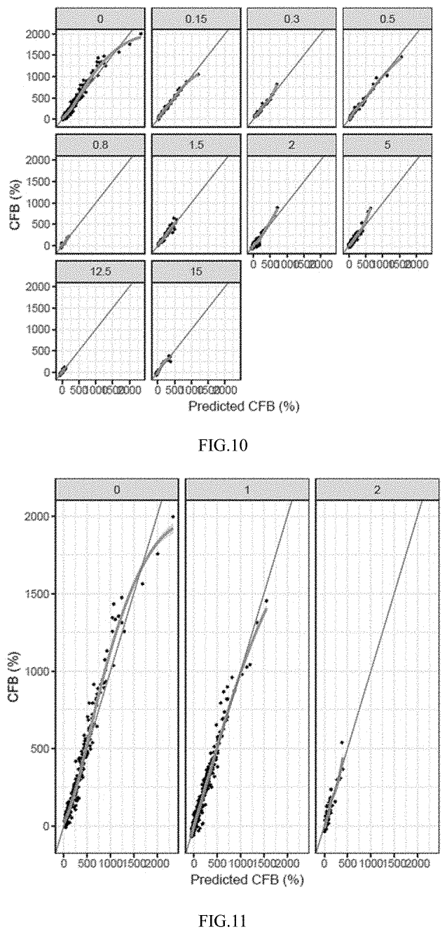

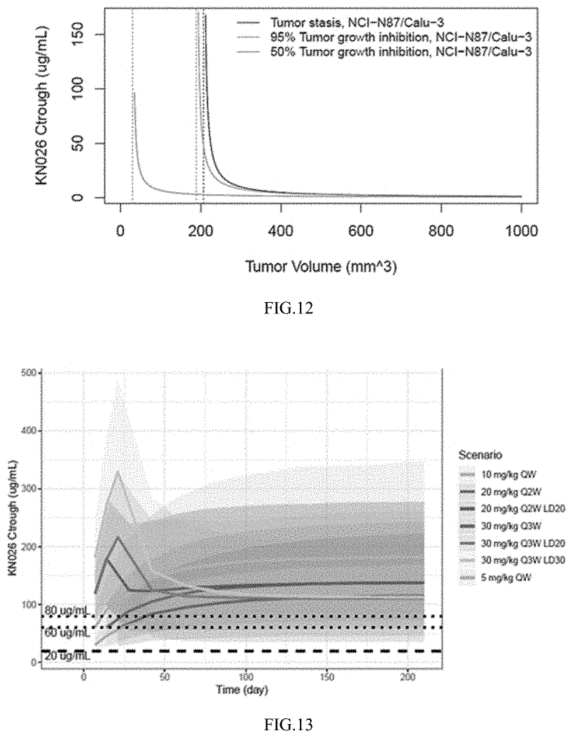

A preliminary ER analysis was performed using 66 post-dose SLD observations from 24 breast cancer patients in the ongoing FIE study (FIG. 6). Among the 24 patients, 14 have only one post-dose SLD observation in addition to the baseline observation, and the rest have more than one post-dose SLD observations (FIG. 7). Currently, an individual patient in the ER dataset has at most 5 post-dose SLD observations.

Due to the sparsity of the interim data, tumor growth rate constant could not be reliably estimated. On the other hand, for a HER2-positive breast cancer patient who has failed at least one prior line of trastuzumab-based therapy, RECIST 1.1 defined progressive disease is usually observed at 6 to 8 weeks apart if remain untreated by an effective therapy. It indicates a 20% increase in from baseline at approximately 8 weeks. Therefore, tumor growth rate constant in the current model was fixed to 0.0228 per week, derived from an empirical growth of 20% within 8 weeks. Steady state Cₘᵢₙ, Cₘₐₓ, and Cₐᵥₑ were tested as the exposure predictor for the tumor response in the ER analysis, and steady state Cₘₐₓ was found to yield the best prediction power based on the current dataset. Using Cₘₐₓ as the exposure metric to drive efficacy on SLD, tumor killing rate constant was estimated to be 0.0943 mL/mg per week with an acceptable precision (RSE=22.6%). The variance of the inter-individual variability on the tumor killing rate constant was estimated to be more than 100%, suggesting a large variability on HER2 bispecific antibody effect within the 24 patients. Simulations based on the interim ER model for SLD suggest that HER2 bispecific antibody doses of 20 or 30 mg/kg will be needed to achieve 30% tumor shrinkage in more than half of the simulated individuals (FIG. 5). Also, more frequent loading doses will have the advantage of maximizing initial tumor killing.

### Dosage Choose

The translational tumor growth inhibition model showed that predicted HER2 bispecific antibody trough concentrations up to 20 μg/mL can noticeably reduce the time to tumor stasis, whereas there seems to be a very limited gain in further tumor growth inhibition when HER2 bispecific antibody trough concentrations higher than 20 μg/mL. On the other hand, it is acknowledged that the current translational tumor growth inhibition model did not take into consideration of variability or uncertainly in any of the parameters. Therefore, this trough concentration of 20 μg/mL probably provides only a rough reference rather than an accurate threshold for the antitumor activity of HER2 bispecific antibody. Based on this analysis, clinical regimens in first in human study were selected to reach C_{trough} of 20 μg/mL while give enough range to explore whether anti-tumor activity of HER2 bispecific antibody was dependent on maintaining minimal target concentration (C_{min,ss} driven), on peak level (C_{max,ss} driven) or on average concentration (C_{avg,ss} or AUC driven) Simulations results from the HER2 bispecific antibody population PK model (Table 4) using data from the FIH study showed that all tested HER2 bispecific antibody doses, ranging from 5 mg/kg QW to 30 mg/kg Q3W, can achieve trough concentrations >20 μg/ml at steady state in almost all subjects (>98% of simulated subjects in each dosing regimen). High disease control rate (66.7%) and long term clinical benefit has been observed in patients at the lowest dose level of 5 mg/kg QW which is in line with the projection from preclinical study. At the same time, preliminary efficacy data from the FIH study seem to show that there is still a gain in SLD response when increasing the dose from 5 or 10 mg/kg QW to 20 mg/kg Q2W or 30 mg/kg Q3W. This probably implies that higher exposure levels are needed to inhibit tumor growth in humans than in xenograft models.

Furthermore, as 10 mg/kg QW regimen has essentially the same average exposure to 20 mg/kg Q2W and 30 mg/kg Q3W regimens, the gain in efficacy with higher dose level but lower frequency seems to suggest that the SLD response is driven more by the peak concentration rather than by the trough or average concentration. Indeed, even with the same level of overall dose amount, the steady state peak concentrations are much higher in 20 or 30 mg/kg dose groups than in 10 mg/kg dose group (Table 4). However, with the limitation that only trough concentrations were collected in the preclinical studies, no direct extrapolation can be established from preclinical or even translational tumor growth inhibition model regarding KC₅₀ and K_{D} associated with peak nor average concentration of HER2 bispecific antibody. Clinical exposure-response analysis further optimizes the understanding toward efficacious dose selection for HER2 bispecific antibody and is complementary to preclinical analysis forming a full course of translational PKPD assessment.

Admittedly, current population PK and exposure-response analysis for SLD were based on a small interim dataset, where only a few patients' data were available in each dosing group. In particular, SLD data from only 3 patients were available for each group of 5 mg/kg QW, 10 mg/kg QW, and 30 mg/kg Q3W. Therefore, current parameters in the SLD exposure-response analysis were estimated with high relative high variability and uncertainty. This is reflected in the simulation results (FIG. 6), where wide ranges of SLD responses were noted for each simulated dosing regimen. With more data becoming available for both PK and efficacy endpoints, it is expected to update and refine both models, and further assess the current assumptions and results.

As a summary, translational PKPD approach well informed the dose selection strategy for HER2 bispecific antibody. 20 mg/kg Q2W and 30 mg/kg Q3W are selected as RP2Ds for future studies. Preliminary exposure-efficacy analysis in humans suggests a potential Cₘₐₓ-driven anti-tumor activity. This observation will be validated by upcoming clinical efficacy data. It is demonstrated that the application of modeling and simulation methods and translational PK-PD approach is a powerful tool to support drug development and improve dose selection strategy for novel bispecific antibody.

### Example 5 Results from the Clinical Trials

### 5.1 Patient Characteristics and Treatment

Patient baseline characteristics for each schedule are listed in Table 6. A total of 63 female patients (median age, 54 years; range, 31 to 69 years) were enrolled from September 2018 to December 2019. Most of the patients were heavily pretreated, with the median number of prior therapeutic lines 3 (range, 1 to 12) and anti-HER2 therapeutic lines 2 (range, 1 to 10) administered in the metastatic setting. Among all, 57.1% patients (36/63) received ≥3 previous palliative treatments. Trastuzumab was previously used in almost all of the patients (61/63, 96.8%), and HER2 TKI and HER2 ADC treatments were also applied in 50.8% (32/63) and 23.8% (15/63) of the patients, respectively. It should be noted that not until December 2018 and February 2020, pertuzumab and T-DM1 were approved in China respectively. Therefore, patients who were not pretreated with pertuzumab or T-DM1 were permitted to be included in this study. Sixty patients (95.2%) had visceral disease at baseline. Common sites of metastases included lung (35 cases, 55.6%), and liver (18 cases, 28.6%). By the time of the data cutoff date for this report, May 22, 2020, 27 patients remained on the study treatment and 36 patients discontinued treatment due to disease progression (n=35) and treatment related adverse events (TRAEs) (n=1) (FIG. 14). In FIG. 14, T represents trastuzumab; P represents pertuzumab; A represents anti-HER2 ADC; and S represents small molecular anti-HER2 TKI. Median treatment duration for the overall population was 5.6 months (range, 1.0˜18.5 months).

TABLE 6
Baseline Demographic and Clinical Characteristics

| | No. of Patients (%) | | | | |
|---|---|---|---|---|---|
| | 5 mg/kg | 10 mg/kg | 20 mg/kg | 30 mg/kg | |
| | QW | QW | Q2W | Q3W | Total |
| Characteristic | (n = 3) | (n = 3) | (n = 28) | (n = 29) | (N = 63) |
| Median age, years | 56 (45, 57) | 57 (43, 57) | 51 (31,68) | 54 (33, 69) | 54 (31,69) |
| (range) | | | | | |

Histology, WHO classification

| | | | | | |
|---|---|---|---|---|---|
| Ductal | 3 (100%) | 3 (100%) | 28 (100%) | 28 (96.6%) | 62 (98.4%) |
| Lobular | 0 | 0 | 0 | 0 | 0(0) |
| Unknown | 0 | 0 | 0 | 1 (3.4%) | 1 (1.6%) |

Hormone receptor

| | | | | | |
|---|---|---|---|---|---|
| ER or PR positive | 1 (33.3%) | 1 (33.3%) | 12 (42.9%) | 12 (41.4%) | 26 (41.3%) |
| ER and PR negative | 2 (66.7%) | 2 (66.7%) | 16 (57.1%) | 17 (58.6%) | 37 (58.7%) |

ECOG performance status

| | | | | | |
|---|---|---|---|---|---|
| 0 | 0 | 0 | 6 (21.4%) | 11 (37.9%) | 17 (27.0%) |
| 1 | 3 (100%) | 3 (100%) | 22 (78.6%) | 18 (62.1%) | 46 (73.0%) |

Prior treatment

| | | | | | |
|---|---|---|---|---|---|
| Neo/Adjuvant setting | 3 (100%) | 2 (66.7%) | 20 (71.4%) | 22 (75.9%) | 47 (74.6%) |
| Anthracyclines | 3 (100%) | 2 (66.7%) | 18 (64.3%) | 21 (72.4%) | 44 (69.8%) |
| Taxanes | 2 (66.7%) | 2 (66.7%) | 18 (64.3%) | 19 (65.5%) | 41 (65.1%) |
| Trastuzumab | 1 (33.3%) | 1 (33.3%) | 11 (39.3%) | 10 (34.5%) | 23 (36.5%) |

Lines in the metastatic setting

| | | | | | |
|---|---|---|---|---|---|
| Median (Range) | 8 (4, 10) | 3 (3, 12) | 3 (1, 10) | 2 (1, 7) | 3 (1, 12) |
| 1 | 0 | 0 | 4 (14.3%) | 6 (20.7%) | 10 (15.9%) |
| 2 | 0 | 0 | 7 (25.0%) | 10 (34.5%) | 17 (27.0%) |
| 23 | 3 (100%) | 3 (100%) | 17 (60.7%) | 13 (44.8%) | 36 (57.1%) |

Anti-HER2 lines in the metastatic setting

| | | | | | |
|---|---|---|---|---|---|
| Median (Range) | 7 (3, 9) | 2 (2, 10) | 2 (1, 9) | 2 (1, 4) | 2 (1, 10) |
| Trastuzumab ± | 3 (100%) | 3 (100%) | 28 (100%) | 27 (93.1%) | 61 (96.8%) |
| Pertuzumab | | | | | |
| Anti-HER2 ADC | 2 (66.7%) | 1 (33.3%) | 7 (25.0%) | 5 (17.2%) | 15 (23.8%) |
| Anti-HER2 TKI | 3 (100%) | 2 (66.7%) | 13 (46.4%) | 14 (48.3%) | 32 (50.8%) |

Abbreviations:
ECOG, Eastern Cooperative Oncology Group performance;
ER, Estrogen or receptor;
PR, Progesterone receptor

### 5.2 Safety

All patients were evaluated for safety assessments (Table 7). No DLTs were observed in all four dose levels. the HER2 bispecific antibody TRAEs of any grade were observed in 54 patients (85.7%) in the total cohort. The most common (≥10%) TRAEs were pyrexia (23.8%), diarrhea (22.2%), aspartate aminotransferase increased (22.2%), alanine aminotransferase increased (22.2%), white blood cell count decreased (15.9%), hypokalemia (12.7%), infusion related reaction (12.7%) and neutrophil count decreased (12.7%). All pyrexia events are considered as AEs related to infusion according to investigator's assessment. And all patients with TRAEs <grade 3 restored well with symptomatic treatments. In total, four patients (6.3%) (two patients in 20 mg/kg Q2W cohort and two patients in 30 mg/kg Q3W cohort) reported grade 3 TRAEs, including infusion related reaction, transaminases increased, ventricular arrhythmia and cardiac myxoma. No grade 4 or 5 AEs were reported. TRAEs leading to treatment discontinuation occurred in one patient (1.5%) allocated in the 20 mg/kg Q2W cohort. The patient had an abnormal ECG when entering the trial, with a prior medication history of using anthracycline and taxanes. After receiving two doses of the HER2 bispecific antibody treatment, ECG indicated ventricular premature beats (quadruple rhythm). The patient was hospitalized and the HER2 bispecific antibody was discontinued. At the 30th day of safe follow-up, the patient recovered. No dose delays or dose reduction were reported.

TABLE 7
Most Common Treatment Related Adverse Events (Any and Grade 3/4)
That Occurred in 10% or More Patients (Safety Analysis Set)

| | 5 mg/kg QW | | 10 mg/kg QW | | 20 mg/kg Q2W | | 30 mg/kg Q3W | | Total | |
|---|---|---|---|---|---|---|---|---|---|---|
| | (N = 3) | | (N = 3) | | (N = 28) | | (N = 29) | | (N = 63) | |
| | Grade ≥3 | Total | Grade ≥3 | Total | Grade ≥3 | Total | Grade ≥3 | Total | Grade ≥3 | Total |
| Subjects with at least 1 | 0 | 3 | 0 | 2 | 2 | 25 | 2 | 24 | 4 | 54 |
| the HER2 bispecific | | (100%) | | (66.7%) | (7.1%) | (89.3%) | (6.9%) | (82.8%) | (6.3%) | (85.7%) |
| antibody of present | | | | | | | | | | |
| applicaiton related TEAE | | | | | | | | | | |
| Pyrexia | 0 | 1 | 0 | 1 | 0 | 8 | 0 | 5 | 0 | 15 |
| | | (33.3%) | | (33.3%) | | (28.6%) | | (17.2%) | | (23.8%) |
| Diarrhea | 0 | 1 | 0 | 1 | 0 | 6 | 0 | 6 | 0 | 14 |
| | | (33.3%) | | (33.3%) | | (21.4%) | | (20.7%) | | (22.2%) |
| Alanine | 0 | 0 | 0 | 0 | 0 | 8 | 0 | 6 | 0 | 14 |
| aminotransferase | | | | | | (28.6%) | | (20.7%) | | (22.2%) |
| increased | | | | | | | | | | |
| Aspartate | 0 | 0 | 0 | 0 | 0 | 6 | 0 | 8 | 0 | 14 |
| aminotransferase | | | | | | (21.4%) | | (27.6%) | | (22.2%) |
| increased | | | | | | | | | | |
| White blood cell | 0 | 2 | 0 | 0 | 0 | 3 | 0 | 5 | 0 | 10 |
| count decreased | | (66.7%) | | | | (10.7%) | | (17.2%) | | (15.9%) |
| Hypokalemia | 0 | 2 | 0 | 0 | 0 | 2 | 0 | 4 | 0 | 8 |
| | | (66.7%) | | | | (7.1%) | | (13.8%) | | (12.7%) |
| Infusion related | 0 | 0 | 0 | 0 | 0 | 3 | 1 | 5 | 1 | 8 |
| reaction | | | | | | (10.7%) | (3.4%) | (17.2%) | (1.6%) | (12.7%) |
| Neutrophil count | 0 | 1 | 0 | 0 | 0 | 4 | 0 | 3 | 0 | 8 |
| decreased | | (33.3%) | | | | (14.3%) | | (10.3%) | | (12.7%) |
| Atrioventricular | 0 | 1 | 0 | 0 | 0 | 4 | 0 | 1 | 0 | 6 |
| block first degree | | (33.3%) | | | | (14.3%) | | (3.4%) | | (9.5%) |
| Hyperglycemia | 0 | 1 | 0 | 0 | 0 | 4 | 0 | 1 | 0 | 6 |
| | | (33.3%) | | | | (14.3%) | | (3.4%) | | (9.5%) |
| Proteinuria | 0 | 1 | 0 | 1 | 0 | 4 | 0 | 0 | 0 | 6 |
| | | (33.3%) | | (33.3%) | | (14.3%) | | | | (9.5%) |
| Anemia | 0 | 0 | 0 | 1 | 0 | 1 | 0 | 3 | 0 | 5 |
| | | | | (33.3%) | | (3.6%) | | (10.3%) | | (7.9%) |
| Electrocardiogram | 0 | 1 | 0 | 1 | 0 | 3 | 0 | 0 | 0 | 5 |
| T wave abnormal | | (33.3%) | | (33.3%) | | (10.7%) | | | | (7.9%) |
| Hypertriglyceridemia | 0 | 0 | 0 | 0 | 0 | 4 | 0 | 1 | 0 | 5 |
| | | | | | | (14.3%) | | (3.4%) | | (7.9%) |
| Rash | 0 | 0 | 0 | 0 | 0 | 3 | 0 | 2 | 0 | 5 |
| | | | | | | (10.7%) | | (6.9%) | | (7.9%) |
| Asthenia | 0 | 1 | 0 | 0 | 0 | 3 | 0 | 0 | 0 | 4 |
| | | (33.3%) | | | | (10.7%) | | | | (6.3%) |
| Blood creatinine | 0 | 2 | 0 | 0 | 0 | 2 | 0 | 0 | 0 | 4 |
| increased | | (66.7%) | | | | (7.1%) | | | | (6.3%) |
| Cough | 0 | 0 | 0 | 0 | 0 | 3 | 0 | 0 | 0 | 3 |
| | | | | | | (10.7%) | | | | (4.8%) |

### 5.3 PK and Immunogenicity Assessments

Single and multiple dose pharmacokinetics following intravenous infusion and dose proportionality of the HER2 bispecific antibody had been characterized in 12 Chinese subjects treated with dose escalation cohort of the first in human study the HER2 bispecific antibody-CHN-001 (data cutoff as of September 2019) by standard non-compartmental analysis based on intensive concentration data obtained over a complete dosing interval of one week, two weeks or three weeks. The exposure parameters of the HER2 bispecific antibody in terms of maximum concentration (Cₘₐₓ) and area under the concentration time curve (AUC_{0-inf}) after first dose generally increased in an approximately dose-proportional manner in the dose range between 5 mg/kg to 30 mg/kg. The total systemic clearance was 19.3 (±5.7) and 14.6 (±4.7) mL/h at 20 mg/kg and 30 mg/kg, respectively. The terminal half-life increased with the dose. The average values were 140 (±23) and 242 (±66) hours for 20 mg/kg and 30 mg/kg doses, respectively. Table 8 displays the main PK parameters that were evaluated after the first and multiple doses of the HER2 bispecific antibody.

Two (3.2%) of the 63 tested patients who were evaluable for post-dose the HER2 bispecific antibody was confirmed positive for antidrug antibodies. No differences were observed in the PK profiles, safety features, or efficacy outcomes for the two patients (data not shown).

TABLE 8
Main PK Parameters after the First Dose and Multiple Doses of the
HER2 bispecific antibody of present application for Subjects

| | Mean ± SD | | | |
|---|---|---|---|---|
| Parameters | AUC_{0-∞} (h · ng/mL) | Cₘₐₓ (ng/mL) | CL (mL/h) | t_{1/2} (h) |
| First dose | | | | |
| 5 mg/kg (n = 3) | 11832249 ± 3321791 | 100351 ± 10644 | 25.3 ± 7.9 | 89 ± 24 |
| 10 mg/kg (n = 3) | 31291034 ± 1952307 | 243669 ± 19174 | 20.9 ± 0.5 | 125 ± 7 |
| 20 mg/kg (n = 3) | 59061158 ± 17515469 | 344933 ± 22727 | 19.3 ± 5.7 | 140 ± 23 |
| 30 mg/kg (n = 3) | 118330657 ± 32957066 | 589587 ± 122731 | 14.6 ± 4.7 | 242 ± 66 |
| Multiple doses | | | | |
| 5 mg/kg (n = 3) | 8610234 ± 1711853 | 10035l ± 10644 | 24.2 ± 8.81 | 180 ± 36 |
| 10 mg/kg (n = 3) | 21459737 ± 4687513 | 243669 ± 19174 | 20.9 ± 0.53 | 166 ± 28 |
| 20 mg/kg (n = 3) | 47666889 ± 11043371 | 344933 ± 22727 | 19.3 ± 5.70 | 298 ± 292 |
| 30 mg/kg (n = 3) | 94436113 ± 21850364 | 589587 ± 122731 | 14.6 ± 4.70 | 203 ± 66.3 |

### 5.4 Clinical Activity

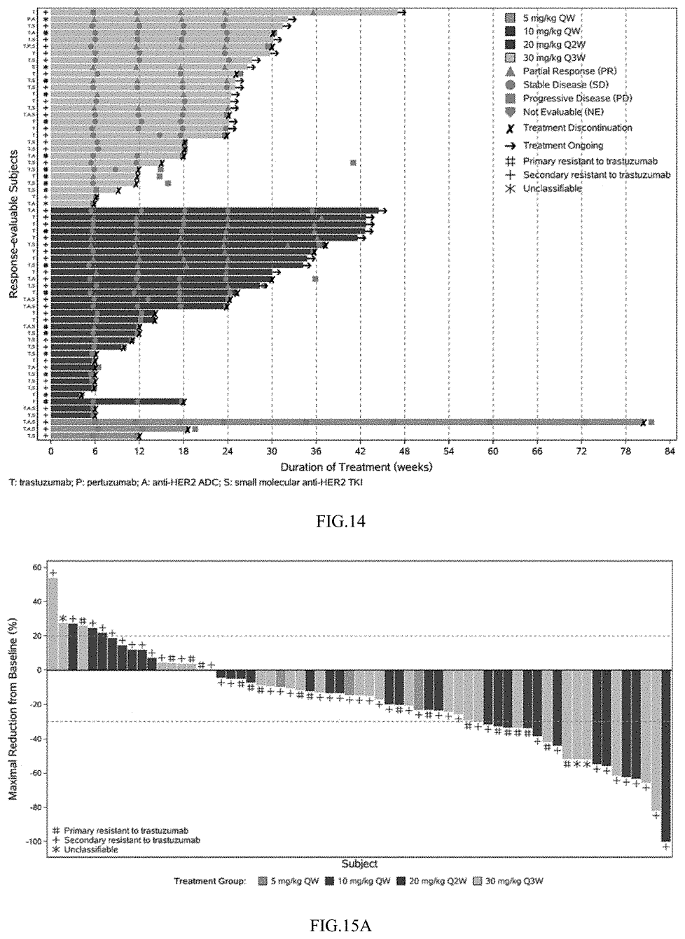

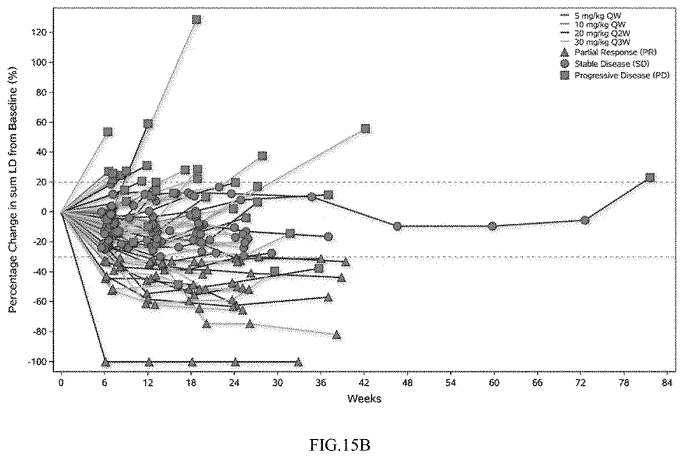

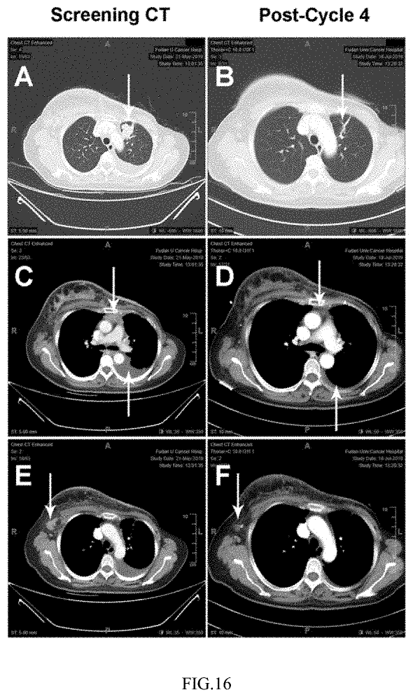

All patients were evaluated for response assessments. With a median follow-up of 8.2 months (range, 4.9˜19.8 months), tumor shrinkage was observed in 46 (73.0%) of 63 patients with measurable lesions who had at least one postbaseline scan (FIG. 15A-15B). FIG. 15A shows maximal change of tumor size from baseline per RECIST v1.1 for patients with at least one post treatment radiographic evaluation. The length of the bar represents maximal decrease or minimal increase in target lesion(s); FIG. 15B shows change of individual tumor burden over time from baseline assessed per RECIST v1.1. Tumor response was assessed before treatment, once every 6 weeks during first 12 months and every 12 weeks thereafter until progressive disease, starting a new anti-tumor therapy or withdrawal of informed consent. In the cohort of recommended phase 2 dose levels (20 mg/kg Q2W and 30 mg/kg Q3W) including 57 patients, a total of 17 (29.8%) achieved a best response of PR, 25 (43.9%) had stable disease (SD), and 14 (24.6%) had progressive disease (PD). The ORR was 29.8% (95% CI, 18.4 to 43.4) and the DCR was 73.7% (95% CI, 60.3 to 84.5). The median DOR was 7.2 months (95% CI, 5.5, NE). The median PFS was 5.6 months (95% CI, 4.2 to 8.2) and the 6-month PFS was 44.6% (95% CI, 29.0 to 59.0). The 12-month PFS and OS were not reached. In the total cohort of 63 evaluable patients, a total of 17 (27.0%) achieved a best response of PR, 28 (44.4%) had SD, and 17 (27.0%) had PD. The ORR was 27.0% (95% CI, 16.6 to 39.7) and the DCR was 71.4% (95% CI, 58.7 to 82.1). The median PFS was 5.5 months (95% CI, 4.1 to 7.0) and the 6-month PFS was 42.0% (95% CI, 27.8 to 55.6) (FIG. 14 and Table 9). Interestingly, the two patients previously treated with pertuzumab both achieved PR after the HER2 bispecific antibody treatment. For example, one of the responders, who had the relapsed breast cancer, previously received adjuvant chemotherapy and radiotherapy (DFI=20 months), first-line docetaxel/trastuzumab/pertuzumab (PFS=10 months) and second-line capecitabine/lapatinib (PFS=7 months). The PFS of third-line the HER2 bispecific antibody (30 mg/kg Q3W) for the patients with PR was 6.77 months (FIG. 16A-16F). In FIGS. 16, the patient was previously treated with adjuvant chemotherapy and radiotherapy, first-line docetaxel/trastuzumab/pertuzumab and second-line capecitabine/lapatinib. There was a significant reduction in tumor size in cycle 4 of third-line the HER2 bispecific antibody of present application (30 mg/kg q3w). Left lung (FIG. 16A, FIG. 16B), anterior mediastinal lymph nodes (FIG. 16C, FIG. 16D) and right axillary lymph nodes (FIG. 16E, FIG. 16F) were included in the target lesions. Left pleural effusion (FIG. 16C, FIG. 16D) was the non-target lesion. The PFS for the patients was 6.77 months.

The efficacy categorized by type of resistance to trastuzumab6, hormone receptor status, and administration or not of the pertuzumab, anti-HER2 TKI or anti-HER2 ADC in the cohort of recommended phase 2 dose levels was summarized in Table 10. In detail, trastuzumab primary resistance is defined as progression at first radiological reassessment at 8-12 weeks or within 3 months after trastuzumab with or without chemotherapy in the metastatic setting or new recurrences diagnosed during or within 12 months after adjuvant trastuzumab. Trastuzumab secondary resistance is defined as disease progression after trastuzumab-containing regimens that initially achieved disease response or stabilization at first radiological assessment. In the total cohort of 63 evaluable patients, the categorized efficacy was summarized in Table 11.

TABLE 9
Best ORR in the total cohort and the cohort of recommended phase 2 dose levels (20 mg/kg Q2W and 30 mg/kg Q3W)

| | | | | | | 20 mg/kg Q2W + |
|---|---|---|---|---|---|---|
| | 5 mg/kg | 10 mg/kg | 20 mg/kg | 30 mg/kg | | 30 mg/kg Q3W |
| | QW | QW | Q2W | Q3W | Total | pooling |
| Characteristic | (n = 3) | (n = 3) | (n = 28) | (n = 29) | (N = 63) | (n = 57) |
| CR (%) | 0 | 0 | 0 | 0 | 0 | 0 |
| PR (%) | 0 | 0 | 10 (35.7%) | 7 (24.1%) | 17 (27.0%) | 17 (29.8%) |
| SD (%) | 2 (66.7%) | 1 (33.3%) | 8 (28.6%) | 17 (58.6%) | 28 (44.4%) | 25 (43.9%) |
| PD (%) | 1 (33.3%) | 2 (66.7%) | 9 (32.1%) | 5 (17.2%) | 17 (27.0%) | 14 (24.6%) |
| NE (%) | 0 | 0 | 1 (3.6%) | 0 | 1 (1.6%) | 1 (1.8%) |
| ORR (95% CI) | 0 | 0 | 35.7% (18.6%, | 24.1% (10.3%, | 27.0% (16.6%, | 29.8% (18.4%, |
| | | | 55.9%) | 43.5%) | 39.7%) | 43.4%) |
| DCR (95% CI) | 66.7% (9.4%, | 33.3% (0.8%, | 64.3% (44.1%, | 82.8% (64.2%, | 71.4% (58.7%, | 73.7% (60.3%, |
| | 99.2%) | 90.6%) | 81.4%) | 94.1%) | 82.1%) | 84.5%) |
| CBR (95% CI) | 33.3% (0.8%, | 0 | 39.3% (21.5%, | 27.6% (12.7%, | 31.7% (20.6%, | 33.3% (21.4%, |
| | 90.6%) | | 59.4%) | 47.2%) | 44.7%) | 47.1%) |
| Median PFS (months, | 4.5 (2.7, | 1.3 (1.3, | 5.5 (2.5, | 5.9 (4.2, | 5.5 (4.1, | 5.6 (4.2, |
| 95% CI) | NE) | NE) | 8.2) | 7.0) | 7.0) | 8.2) |

Abbreviations: CBR, clinical benefit rate (CR + PR + SD 24 weeks); CR, complete response; DCR, disease control rate (CR + PR + SD); ORR, objective response rate (CR + PR); PD, progressive disease; PFS, progression-free survival; PR, partial response; SD, stable disease.

TABLE 10
The efficacy categorized by different characteristics
in the cohort of recommended phase 2 dose levels

| | | | | | | | | Months |
|---|---|---|---|---|---|---|---|---|
| | | | | | | | | (95% CI) |
| | No. of Patients (%) | | | | Rate (95% CI) | | | Median PFS |
| Characteristic | CR | PR | SD | PD | ORR | DCR | CBR | (months) |
| Total (N = 57) | 0 | 17 | 25 | 14 | 17 | 42 | 19 | 5.6 |
| | | (29.8%) | (43.9%) | (24.6%) | (29.8%) | (73.7%) | (33.3%) | (4.2, 8.2) |

Hormone receptor

| | | | | | | | | |
|---|---|---|---|---|---|---|---|---|
| ER or PR positive | 0 | 7 | 10 | 6 | 7 | 17 | 7 | 5.5 |
| (n = 24) | | (29.2%) | (41.7%) | (25.0%) | (29.2%) | (70.8%) | (29.2%) | (2.7, 8.4) |
| ER and PR negative | 0 | 10 | 15 | 8 | 10 | 25 | 12 | 5.6 |
| (n = 33) | | (30.3%) | (45.4%) | (24.2%) | (30.3%) | (75.8%) | (36.4%) | (4.2, NE) |

Resistance to trastuzumab

| | | | | | | | | |
|---|---|---|---|---|---|---|---|---|
| Primary resistance | 0 | 4 | 5 | 7 | 4 | 9 | 4 | 3.9 |
| (n = 17) | | (23.5%) | (29.4%) | (41.2%) | (23.5%) | (52.9%) | (23.5%) | (2.7, 7.0) |
| Secondary | 0 | 11 | 20 | 6 | 11 | 31 | 13 | 6.8 |
| resistance (n = 37) | | (29.7%) | (54.0%) | (16.2%) | (29.7%) | (83.8%) | (35.1%) | (5.4, 8.4) |
| Unclassifiable | 0 | 2 | 0 | 1 | 2 | 2 | 2 | NE |
| (n = 3) | | (66.7%) | | (33.3%) | (66.7%) | (66.7%) | (66.7%) | (1.31, NE) |

Previous anti-HER2 therapy

| | | | | | | | | |
|---|---|---|---|---|---|---|---|---|
| Pertuzumab (n = 2) | 0 | 2 | 0 | 0 | 2 | 2 | 2 | 6.8 |
| | | (100%) | | | (100%) | (100%) | (100%) | (NE, NE) |
| Anti-HER2 ADC | 0 | 1 | 7 | 3 | 1 | 8 | 2 | 5.5 |
| (n = 11) | | (9.1%) | (63.6%) | (27.3%) | (9.1%) | (72.7%) | (18.2%) | (1.5, NE) |
| Anti-HER2 TKI | 0 | 7 | 12 | 9 | 7 | 19 | 8 | 5.5 |
| (n = 28) | | (25.0%) | (42.8%) | (32.1%) | (25.0%) | (67.8%) | (28.6%) | (2.7, 6.8) |

Abbreviations: ER, Estrogen or receptor; PR, Progesterone receptor; CBR, clinical benefit rate (CR + PR + SD ≥ 24 weeks); CR, complete response; DCR, disease control rate (CR + PR + SD); ORR, objective response rate (CR + PR); PD, progressive disease; PFS, progression-free survival; PR, partial response; SD, stable disease.

TABLE 11
The efficacy categorized by different characteristics in the total cohort of 63 evaluable patients

| | | | | | | | | Months |
|---|---|---|---|---|---|---|---|---|
| | | | | | | | | (95% CI) |
| | No. of Patients (%) | | | | Rate (95% CI) | | | Median PFS |
| Characteristic | CR | PR | SD | PD | ORR | DCR | CBR | (months) |
| Total (N = 63) | 0 | 17 | 28 | 17 | 17 | 45 | 20 | 5.5 |
| | | (27.0%) | (44.4%) | (27.0%) | (27.0%) | (71.4%) | (31.7%) | (4.1, 7.0) |

Hormone receptor

| | | | | | | | | |
|---|---|---|---|---|---|---|---|---|
| ER or PR positive | 0 | 7 | 11 | 7 | 7 | 18 | 8 | 5.5 |
| (n = 26) | | (26.9%) | (42.3%) | (26.9%) | (26.9%) | (69.2%) | (30.8%) | (2.7, 8.4) |
| ER and PR negative | 0 | 10 | 17 | 10 | 10 | T1 | 12 | 5.5 |
| (n = 37) | | (27.0%) | (45.9%) | (27.0%) | (27.0%) | (73.0%) | (32.4%) | (4.1, 8.2) |

Resistance to trastuzumab

| | | | | | | | | |
|---|---|---|---|---|---|---|---|---|
| Primary resistance | 0 | 4 | 6 | 7 | 4 | 10 | 4 | 4.1 |
| (n = 18) | | (22.2%) | (33.3%) | (38.9%) | (22.2%) | (55.6%) | (22.2%) | (2.7, 7.0) |
| Secondary resistance | 0 | 11 | 22 | 9 | 11 | 33 | 14 | 5.9 |
| (n = 42) | | (26.2%) | (52.4%) | (21.4%) | (26.2%) | (78.6%) | (33.3%) | (4.2, 8.2) |
| Unclassifiable (n = 3) | 0 | 2 | 0 | 1 | 2 | 2 | 2 | NE |
| | | (66.7%) | (0.0%) | (33.3%) | (66.7%) | (66.7%) | (66.7%) | (1.31, NE) |

Previous anti-HER2 therapy

| | | | | | | | | |
|---|---|---|---|---|---|---|---|---|
| Pertuzumab (n = 2) | 0 | 2 | 0 | 0 | 2 | 2 | 2 | 6.8 |
| | | (100%) | | | (100%) | (100%) | (100%) | (NE, NE) |
| Anti-HER2 ADC | 0 | 1 | 9 | 4 | 1 | 10 | 3 | 5.5 |
| (n = 14) | | (7.1%) | (64.3%) | (28.6%) | (7.1%) | (71.4%) | (21.4%) | (1.5, 7.0) |
| Anti-HER2 TKI | 0 | 7 | 14 | 12 | 7 | 21 | 7 | 4.5 |
| (n = 33) | | (21.2%) | (42.4%) | (36.4%) | (21.2%) | (63.6%) | (21.2%) | (2.7, 6.8) |

Abbreviations: ER, Estrogen or receptor; PR, Progesterone receptor; CBR, clinical benefit rate (CR + PR + SD ≥ 24 weeks); CR, complete response; DCR, disease control rate (CR + PR + SD); ORR, objective response rate (CR + PR); PD, progressive disease; PFS, progression-free survival; PR, partial response; SD, stable disease.

From the results of the example 5, the recommended Phase 2 doses of the HER2 bispecific antibody of the present application may be 20 mg/kg Q2W and 30 mg/kg Q3W based on safety, clinical responses and pharmacokinetic parameters. The safety of the HER2 bispecific antibody of the present application may have both similarities and differences with trastuzumab and pertuzumab. And the HER2 bispecific antibody of the present application may have a comparable antitumor effect with the treatment combined with trastuzumab and pertuzumab; and may have promising results and all the patients previously treated with pertuzumab achieved PR. The HER2 bispecific antibody of the present application may be well tolerated and has demonstrated encouraging anti-tumor activity in HER2-positive breast cancer patients who have failed anti-HER2 therapies.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method of preventing, alleviating or treating tumor or inhibiting tumor growth in a subject, comprising: administrating to the subject a dose of about 15 mg/kg to about 35 mg/kg of a HER2 bispecific antibody,
wherein said HER2 bispecific antibody comprises a first light chain, a second light chain, a first heavy chain and a second heavy chain,
wherein said first light chain and said second light chain is capable of assembling with a heavy chain of Pertuzumab and a heavy chain of Trastuzumab, respectively;
wherein variable region of said first light chain and/or said second light chain comprises an amino acid sequence as set forth in any one of SEQ ID NO: 1-6.

2. (canceled)

3. (canceled)

4. The method according to claim 1, wherein said first light chain comprises an amino acid sequence as set forth in any one of SEQ ID NO: 7-12, and/or, said second light chain comprises an amino acid sequence as set forth in any one of SEQ ID NO: 7-12.

5. (canceled)

6. The method according to claim 1, wherein variable region of said first heavy chain comprises an amino acid sequence as set forth in SEQ ID NO: 13 and variable region of said second heavy chain comprises an amino acid sequence as set forth in SEQ ID NO: 14.

7. (canceled)

8. (canceled)

9. The method according to claim 1, wherein said first heavy chain or said second heavy chain comprises a sequence as set forth in any one of SEQ ID NO: 15-18.

10. The method according to claim 1, wherein said dose is about 20 mg/kg to about 30 mg/kg.

11. (canceled)

12. (canceled)

13. The method according to claim 1, wherein said HER2 bispecific antibody is administrated once every two weeks or once every three weeks.

14. (canceled)

15. (canceled)

16. The method according to claim 1, wherein said subject was not responsive to a conventional therapy for HER2-related tumor.

17. (canceled)

18. (canceled)

19. The method according to claim 1, wherein said tumor comprises solid tumor.

20. The method according to claim 1, wherein said tumor comprises metastatic tumor, early tumor and/or locally advanced tumor.

21. (canceled)

22. The method according to claim 1, wherein said tumor comprises a breast cancer and/or a gastric cancer.

23. The method according to claim 22, wherein said breast cancer comprises HER2 positive breast cancer and/or HER2 low-expression breast cancer.

24. The method according to claim 22, wherein said breast cancer comprises early breast cancer, locally advanced breast cancer and/or metastatic breast cancer; and/or said gastric cancer comprises early gastric cancer, locally advanced gastric cancer and/or metastatic gastric cancer.

25. (canceled)

26. A formulation for use in preventing, alleviating or treating tumor or inhibiting tumor growth in a subject in need of, the formulation comprises at least 5 μg/mL of a HER2 bispecific antibody,
wherein said HER2 bispecific antibody comprises a first light chain, a second light chain, a first heavy chain and a second heavy chain,
wherein said first light chain and said second light chain is capable of assembling with a heavy chain of Pertuzumab and a heavy chain of Trastuzumab, respectively;
wherein variable region of said first light chain and/or said second light chain comprises an amino acid sequence as set forth in any one of SEQ ID NO: 1-6.

27. (canceled)

28. (canceled)

29. The formulation according to claim 26, wherein said first light chain comprises an amino acid sequence as set forth in any one of SEQ ID NO: 7-12, and/or, said second light chain comprises an amino acid sequence as set forth in any one of SEQ ID NO: 7-12.

30. (canceled)

31. The formulation according to claim 26, wherein variable region of said first heavy chain comprises an amino acid sequence as set forth in SEQ ID NO: 13; and variable region of said second heavy chain comprises an amino acid sequence as set forth in SEQ ID NO: 14.

32. (canceled)

33. (canceled)

34. The formulation according to claim 26, wherein said first heavy chain or said second heavy chain comprises a sequence as set forth in any one of SEQ ID NO: 15-18.

35. (canceled)

36. (canceled)

37. (canceled)

38. A drug delivery device for use in preventing, alleviating or treating tumor or inhibiting tumor growth in a subject in need of, comprises a formulation comprising at least 5 μg/mL of a HER2 bispecific antibody,
wherein said HER2 bispecific antibody comprises a first light chain, a second light chain, a first heavy chain and a second heavy chain,
wherein said first light chain and said second light chain is capable of assembling with a heavy chain of Pertuzumab and a heavy chain of Trastuzumab, respectively;
wherein variable region of said first light chain and/or said second light chain comprises an amino acid sequence as set forth in any one of SEQ ID NO: 1-6.

39. (canceled)

40. (canceled)

41. The drug delivery device according to claim 38, wherein said first light chain comprises an amino acid sequence as set forth in any one of SEQ ID NO: 7-12, and/or, said second light chain comprises an amino acid sequence as set forth in any one of SEQ ID NO: 7-12.

42. (canceled)

43. The drug delivery device according to claim 38, wherein variable region of said first heavy chain comprises an amino acid sequence as set forth in SEQ ID NO: 13; and variable region of said second heavy chain comprises an amino acid sequence as set forth in SEQ ID NO: 14.

44. (canceled)

45. (canceled)

46. The drug delivery device according to claim 38, wherein said first heavy chain or said second heavy chain comprises a sequence as set forth in any one of SEQ ID NO: 15-18.

47. (canceled)

48. (canceled)

49. (canceled)
